SEQUENCE LISTING (3 sequences)

SEQ ID NO: 1 - DNA, 12 bp, Artificial Sequence
  synthetic oligonucleotide
cggnagctyccg

SEQ ID NO: 2 - DNA, 12 bp, Artificial Sequence
  synthetic oligonucleotide
cttttcnttctt

SEQ ID NO: 3 - DNA, 12 bp, Artificial Sequence
  synthetic oligonucleotide
cggnagctyccg

(19) United States
(12) Patent Application Publication
(10) Pub. No.: US 2006/0154889 A1
(43) Pub. Date: Jul. 13, 2006

(54) Polyfluorinated nucleoside analogs and methods of use thereof

(75) Inventor: Eric Todd Kool, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 11/295,306

(22) Filed: Dec. 5, 2005

Related U.S. Application Data
(60) Provisional application No. 60/635,232, filed on Dec. 10, 2004.

(51) Int. Cl. A61K 48/00; C07H 21/04; C07H 19/04

(52) U.S. Cl. 514/44; 536/26.2; 536/23.2

(57) ABSTRACT

Nucleoside analogs comprising polyfluorinated benzene, pyrrole, pyridine, indole, isoindole, or benzoimidazole rings are shown to provide for selective base pairing with self, or other polyfluorinated analogs, in preference to native nucleic acid bases. The analogs of the invention stabilize the stacking of helices, and increase hydrophobicity when introduced into an oligonucleotide.

This invention was made with Government support under contract EB002059 awarded by the National Institutes of Health. The Government has certain rights in this invention.

### BACKGROUND OF THE INVENTION

The physical and chemical factors that allow DNA to perform its functions in the cell have been studied for several decades. Recent advances in the synthesis and manipulation of DNA have allowed this field to move ahead especially rapidly during the past fifteen years. One of the most common chemical approaches to the study of interactions involving DNA has been the use of DNA base analogs in which functional groups are added, deleted, blocked, or rearranged.

Considerable research is being directed to the application of oligonucleotides and the afore-mentioned oligonucleotide analogs as antisense agents for diagnostics., research reagents and therapeutic compounds. Such nucleoside analogs have been useful as in providing specific properties to oligonucleotide probes for diagnostic applications; to antisense RNA and RNAi; and in the synthesis and purification of oligonucleotides. Other nucleoside analogs are used as metabolic inhibitors of viruses and proliferating cells, including tumor cells. Currently a number of nucleoside based drugs are being used to treat human diseases, including AIDS, against various cancers and for various systemic disease resulting from inappropriate immune responses.

Through selection of appropriate analogs in the sugars, bases and backbone, oligonucleotides can be synthesized to have properties that are tailored for the desired use. These modifications may be designed to enhance to provide stability against nucleases and other enzymes that degrade or interfere with the structure or activity of the oligonucleotides, to provide a mode of disruption once sequence-specifically bound to a target, to improve the pharmacokinetic properties of the oligonucleotides, to assist in purification of the oligonucleotide or an oligonucleotide-target complex, to increase cell penetration; and the like.

One deficiency of native DNA and RNA is a susceptibility to enzymatic degradation by a variety of ubiquitous nucleases. Unmodified, “wild type”, oligonucleotides are not useful as therapeutic agents because they are rapidly degraded by nucleases. Therefore, modification of oligonucleotides for conferring nuclease resistance on them has been a focus of research directed towards the development of oligonucleotide therapeutics and diagnostics.

In addition to nuclease stability, the ability of an oligonucleotide to bind to a specific DNA or RNA with fidelity is a further important factor. While much attention has been focused on natural base pairing and selectivity, alternative pairing chemistries have also been explored (see Kool et al. (2000) Angew. Chem. Int. Ed. 39:990-1009).

Among the uses of oligonucleotides are methods of inhibiting gene expression with antisense oligonucleotides complementary to a specific target messenger RNA (mRNA) sequences. Oligonucleotides also have found use in diagnostic tests performed using biological fluids, tissues, intact cells or isolated cellular components. For diagnostics, oligonucleotides and oligonucleotide analogs can be used in cell free systems, in vitro, ex vivo or in vivo.

Oligonucleotides are also widely used as research reagents. They are useful for understanding the function of many other biological molecules as well as in the preparation of such other biological molecules. For example, oligonucleotides serve as primers in the reactions associated with polymerase chain reaction (PCR), which reactions are now widely used in forensics, paleontology, evolutionary studies and genetic counseling, to name just a few.

The development of useful nucleoside analogs is of great interest for research and clinical purposes. The present invention provides for novel and useful polyfluorinated nucleoside analogs.

### SUMMARY OF THE INVENTION

Compositions of polyfluorinated nucleoside analogs are provided. The analogs of the invention selectively pair with self, or other polyfluorinated analogs, in preference to native nucleic acid bases, thereby providing the means for a unique, non-natural pairing configuration. The analogs of the invention may be used to confer properties of interest to single or double stranded oligonucleotides, including enhanced stability, due to effects on stacking; and increased hydrophobicity. Compositions of interest include glycosides comprising the polyfluorinated nucleoside analogs; mono-, di-, and triphosphate esters thereof; derivatives suitable for in vitro synthetic reactions; and oligonucleotides wherein at least one nucleoside is a polyfluorinated analog of the invention.

### BRIEF DESCRIPTION OF THE DRAWINGS

FIG. 1A-1C: Structures and properties of the seven nucleoside analogues in this study. a) Molecular structures of the nucleobases (all are attached at C1′ of the deoxyribose). b) Calculated electrostatic surface potentials of six progressively fluorinated aromatic base analogues, with an attached methyl group to approximate the effects of the deoxyribose 1′ carbon (red depicts negative potential and blue, positive). c) Calculated dipole moments (debye) of aromatic fluorinated base analogues; dipole orientations are shown with yellow arrows, fluorine atoms are in green. Electrostatics were calculated with Spartan '02 (Wavefunction Inc.) employing the AM1 Hamiltonian.

FIG. 2: Plot showing trends in stacking free energies as a function of the number of fluorine substitutions on the phenyl deoxyribose adjacent to cytosine (black), adenine (red), guanine (green), and thymine (blue). Primary data are given in Table 1 and in Table 2; the 2,4,6-trifluorinated case is omitted here.

FIG. 3A-3C: Testing possible linear relationships between stacking free energies and calculated physical properties of aromatic analogues in two comparative sequences with C adjacent (black) and with A adjacent (red)(see Table 1). a) Number of fluorine substitutions (a rough measure of quadrupole strength). b) Dipole moment m (debye) of the methylated base. c) Estimated surface area of the dangling residue excluded from solvent on stacking. The two compounds with bis-ortho effects were omitted.

FIG. 4A-4B. Structures of fluorinated and hydrocarbon DNA base replacements. (a) Chemical structures of the four nucleosides in the study. (b) Electrostatic surface potentials of bases with methyl groups at the point of attachment to deoxyribose. Calculated with Spartan '02 (Wavefunction, Inc.) using the AM1 Hamiltonian.

FIG. 5. Histogram of base-pair stabilities as measured for double substitution of the pair into a 12-bp duplex (see Table 3).

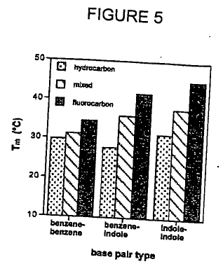

FIG. 6A-6B. A) Melting curves of self-complimentary sequences containing fluorinated base pairs. 5 mM concentration B) Van't Hoff plots of same sequences. Concentrations range from 1 mM to 20 mM.

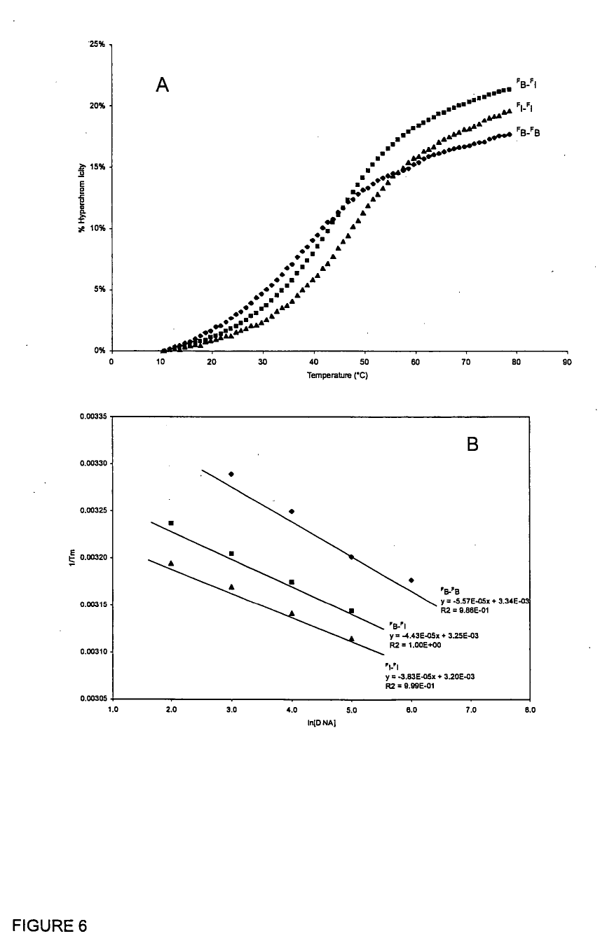

### DETAILED DESCRIPTION OF THE INVENTION

Polyfluorinated nucleoside, nucleotide and oligonucleotide analogs comprising glycosides of polyfluorinated benzene, pyrrole, pyridine, indole, isoindole, or benzoimidazole rings are provided herein. Such polyfluorinated analogs are shown to provide for selective base pairing with self, or other polyfluorinated analogs, in preference to native nucleic acid nucleosides. The analogs of the invention stabilize the stacking of helices, and increase hydrophobicity when introduced into an oligonucleotide. Analogs of the invention may be introduced at any position in an oligonucleotide, e.g. 5′ terminus, 3′ terminus, or internal positions, e.g. through in vitro synthetic reactions. Oligonucleotides may be provided in a single stranded or double stranded form. When present in a double stranded form, the analog bases may be “matched”, i.e. paired with another polyfluorinated analog; “mismatched”, i.e. paired with a naturally occurring base; or overhanging at the 5′ or 3′ terminus.

Compounds of interest include glycosides, nucleosides, nucleotides and oligonucleotides comprising the polyfluorinated nucleoside base analogs. Any of such compounds may be provided in combination with a pharmaceutically acceptable excipient, e.g. for use as an antisense reagent; as a metabolic inhibitor; as a diagnostic probe; and the like.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing those components that are described in the publications that might be used in connection with the presently described invention.

As used herein, compounds which are “commercially available” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.); Molecular Probes (Eugene, Oreg.); Applied Biosystems, Inc. (Foster City, Calif.); and Glen Research (Sterling, Va.).

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified though various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandier et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. The term lower alkyl will be used herein as known in the art to refer to an alkyl, straight, branched or cyclic, of from about 1 to 6 carbons.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts. “Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)— or (S)— or, as (D)- or (L)- for amino acids. The present invention is meant to include all such possible isomers, as well as, their racemic and optically pure forms. Optically active (+) and (−), (R)— and (S)—, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

### Compositions

Polyfluorinated nucleoside analogs of interest are glycosides of tri- and tetra-fluorinated benzene, pyrrole, pyridine, indole, isoindole, or benzoimidazole rings. Also included are derivatives thereof, e.g. nucleotides, oligonucleotides, and the like; which may include various modifications to the sugar and phosphate backbone, as is known in the art. Included in the invention are compounds comprising a structure as set forth below:

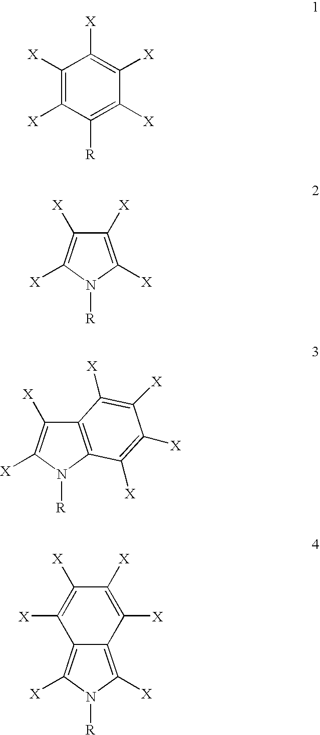

where R is a sugar moiety, including any pentose or hexose sugars, particularly ribose and deoxyribose, which may be in an N-glycoside or C-glycoside configuration, having a D- or L stereochemistry; and

each X is independently selected from H, F, or CF₃, with the proviso that at least 3 and not more than 6 positions have fluorine-containing substituents. For structures 1 and 2, at least 3 and not more than 4 positions will have fluorine-containing substituents. In some embodiments, an annular carbon is substituted with a heteroatom, usually with N.

Some specific compounds of interest include the following glycoside structures:

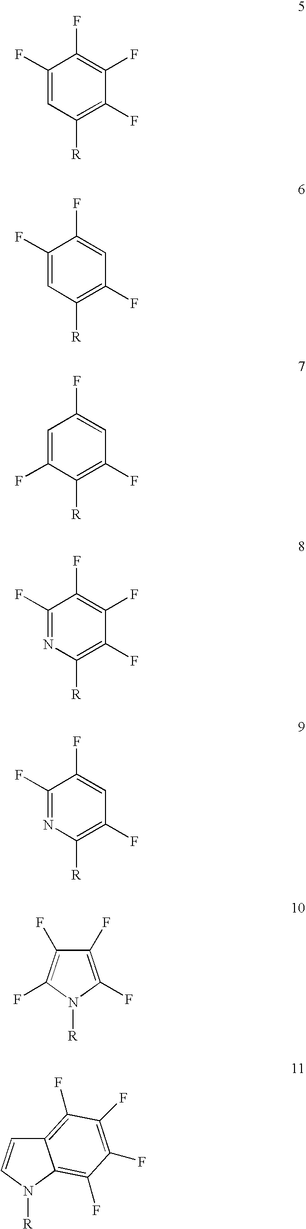

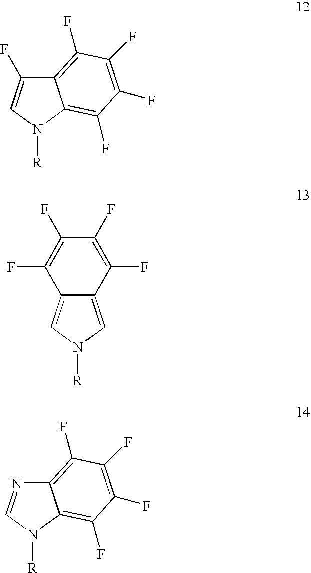

In some embodiments of the invention, the compound is a glycoside of 2,3,4,5-tetrafluorobenzene, or a glycoside of 4,5,6,7-tetrafluoroindole; as well as derivatives thereof.

As used herein, “nucleoside” means a base covalently attached to a sugar or sugar analog and which may contain a phosphate, phosphoroamidite, diphosphate, cyclic phosphate, triphosphate, phosphite or phosphine, where sugars include any pentose or hexose sugars, particularly ribose and deoxyribose, which may be in an N-glycoside or C-glycoside configuration, having a D- or L stereochemistry. Sugar modifications of interest include, without limitation, ribose, deoxyribose and dideoxyribose sugars comprising modifications at the 2′, 3′, 4′ and 5′ positions, particularly where the nucleoside is intended as a pharmaceutical agent, e.g. as an anti-viral or anti-proliferative drug. Some specific examples of modifications at the 2′ and 3′position of sugar moieties are azido, OH, SH, SCH₃, F, OCN, O(CH₂)ₙ NH₂, O(CH₂)ₙ CH₃ where n is from 1 to about 10; C₁ to C₁₀ lower alkyl, substituted lower alkyl, alkaryl or aralkyl; Cl, Br, CN, CF₃, OCF₃, O—, S—, or N-alkyl; O—, S—, or N-alkenyl; SOCH₃, SO₂; CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties.

The pentose moiety can be replaced by a hexose or an alternate structure such as a cyclopentane ring, a 6-member morpholino ring and the like. Nucleosides as defined herein are also intended to include a base linked to an amino acid and/or an amino acid analog having a free carboxyl group and/or a free amino group and/or protected forms thereof.

As used herein, “nucleotide” refers to a nucleoside having a phosphate, triphosphate, or phosphate analog group, particularly a nucleoside in which the 5′-hydroxyl group of a nucleoside is esterified with a phosphate, boranophosphate, or otherwise modified phosphate group.

In some embodiments of the invention, an oligonucleotide comprising one or more of the subject polyfluorinated nucleoside analogs is provided, internally, and/or at either or both of the 5′ or 3′ ends of a linear nucleic acid molecule. The subject base analogs may be present in more than one position in an RNA or DNA molecule, although it is preferred than not more than about 5, usually not more than about 3, more usually not more than about one or two of the analog bases are present in a contiguous “run”. One or more base analogs may be incorporated within a stretch of sequence so that the DNA or RNA fragment is effectively tagged towards the middle of the molecule. The RNA or DNA sequence may comprise a linear, hairpin, dumbbell, circular, or branched conformation and may be single or double stranded. The term “oligonucleotide” refers to a polynucleotide formed from naturally occurring bases, such as purine and pyrimidine heterocycles, and furanosyl groups joined by native phosphodiester bonds, as well as synthetic species formed from naturally occurring subunits or their close homologs, and synthetic species comprising non-naturally occurring analogs (in addition to the polyfluorinated analog(s)) of bases, backbone and/or sugars.

Purines and pyrimidines other than those normally found in nature may included in oligonucleotides. For example, deaza or aza purines and pyrimidines may be used in place of naturally purine or pyrimidine bases and pyrimidine bases having substituent groups at the 5- or 6-positions; purine bases may have altered or replacement substituent groups at the 2-, 6- or 8-positions.

Oligonucleotides may also comprise backbone modifications, including peptide nucleic acids (PNA), locked nucleic acids (LNA), etc., methylations, morpholino derivatives; phosphoroamidate derivatives; unusual base-pairing combinations such as the isobases isocytidine and isoguanidine and the like. Derivatives can also include 3′ and 5′ modifications such as capping.

As is used in the art, the term “oligonucleotide” usually refers to shorter molecules, usually of at least about 3 bases in length, more usually at least 4, 5, or 6 bases; for many embodiments of the invention, preferred oligonucleotides are at least 7 bases, at least 8 bases, at least 10 bases, at least 12 bases, and not more than about 100 bases in length, usually not more than about 50 bases in length, or any length range between any two of these lengths. The term “polynucleotide” may refer to any length of nucleic acid greater than a single base; although in many instances will be used to refer to molecules as present in living organisms, which range from about 50 bases in length to many megabases, in the case of genomic DNAs.

The oligonucleotide can be derived from a completely chemical synthesis process, such as a solid phase mediated chemical synthesis, or from a biological origin, such as through isolation from almost any species that can provide DNA or RNA, or from processes that involve the manipulation of nucleic acids by molecular biology tools, such as DNA replication, PCR amplification, reverse transcription, or from a combination of those processes. Modifications to introduce a polyfluorinated base analog of the invention may be performed post-synthetically; may be used as a primer in a synthetic reaction, e.g. PCR; may be introduced at any position during in vitro synthesis; and the like.

Modified oligonucleotides of the invention may be provided in solution, or bound to a substrate. One, a pair or a plurality of modified probes may be provided in any configuration. By “solid substrate” or “solid support” is meant any surface to which the probes of the invention are attached. A variety of solid supports or substrates are suitable for the purposes of the invention, including both flexible and rigid substrates. By flexible is meant that the support is capable of being bent, folded or similarly manipulated without breakage. Examples of flexible solid supports include nylon, nitrocellulose, polypropylene, polyester films, such as polyethylene terephthalate, etc. Rigid supports do not readily bend, and include glass, fused silica, quartz, acrylamide; plastics, e.g. polytetrafluoroethylene, polypropylene, polystyrene, polystyrene and sulfonated polystyrene-divinyl benzene, quaternized product of chloromethylated polystyrene-divinyl benzene, PEG-polystyrene, PEG, polycarbonate, and blends thereof, and the like; metals, e.g. gold, platinum, silver, and the like; etc. The substrates can take a variety of configurations, including planar surfaces, filters, fibers, membranes, beads, particles, dipsticks, sheets, rods, etc. The substrates can be prepared using any convenient means. One means of preparing the supports is to synthesize the probes, and then deposit them on the support surface. The probes can be deposited on the support using any convenient methodology, including manual techniques, e.g. by micropipette, ink jet, pins, etc., and automated protocols. The probes may also be covalently attached to the substrate, using methods known in the art. Alternatively, the probes can be synthesized on the substrate using standard techniques known in the art.

### Synthetic Methods

Examples of methods for the synthesis of nucleoside compounds according to formulas 1-4 are shown in schemes 1 and 2.

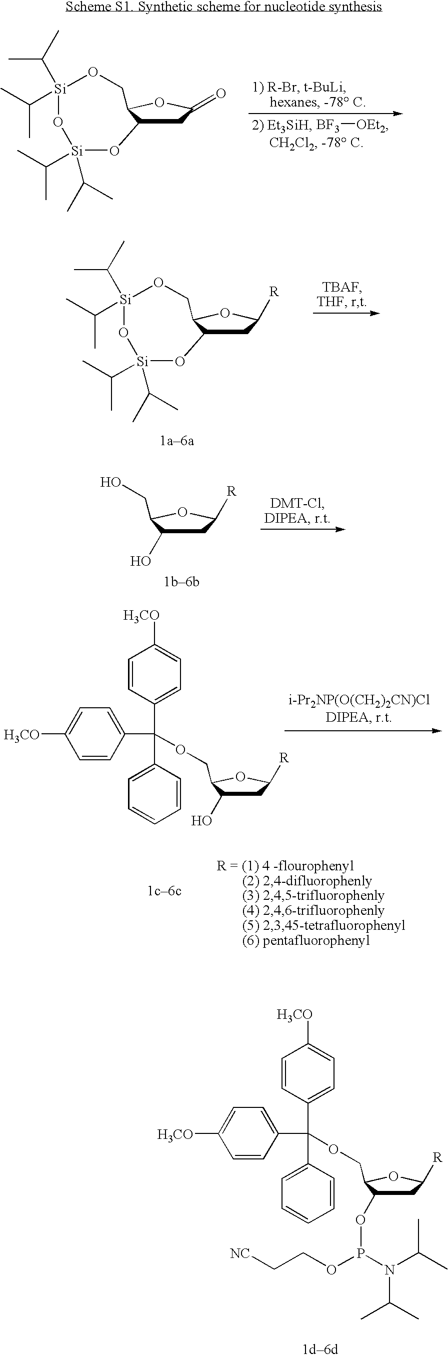

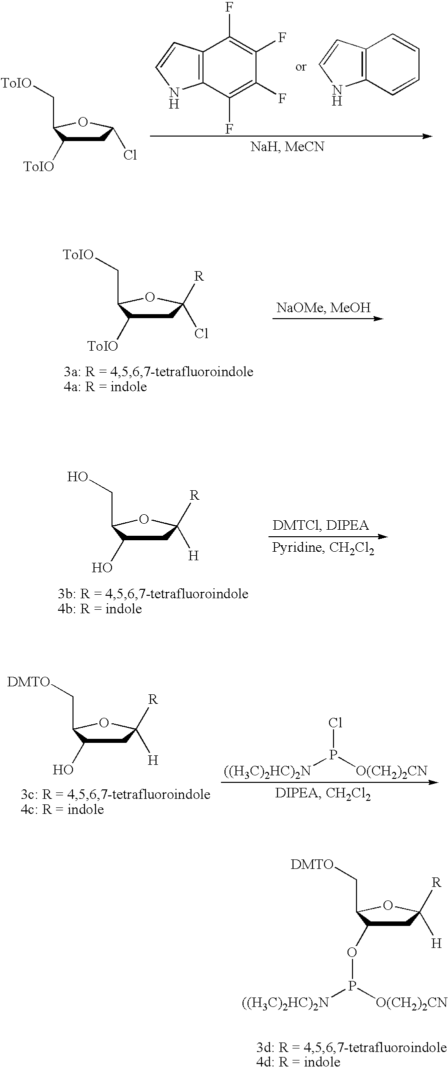

Aryllithium derivatives of the fluorinated aromatic species are reacted with 3′,5′-O-((1,1,3,3-tetraisopropyl)disiloxanediyl)-2′-deoxy-D-ribono-1,4-lactone in hexanes at −78° C. to give a hemiketal which is then reduced to give the protected nucleoside. These couplings give the β-anomer with high selectivity and comparable yields. The siloxane is then deprotected using TBAF to give the free nucleoside. If desired, standard methods may be used to convert the free unprotected nucleosides to 5′-dimethoxytrityl-protected derivatives. These derivatives may then be converted into cyanoethyl phosphoramidite derivatives, and purified by column chromatography.

Alternatively, N-nucleosides are synthesized by coupling the fluorinated aromatic species to 1′-chloro-2′-deoxy-3′,5′-di-O-p-toluoyl-a-D-erythropentofuranose in the presence of sodium hydride. Deprotection of toluoyl groups is performed using sodium methoxide. If desired, standard methods may be used to convert the free unprotected nucleosides to 5′-dimethoxytrityl-protected derivatives. These derivatives may then converted into cyanoethyl phosphoramidite derivatives.

The β-C-deoxynucleosides can be incorporated into oligonucleotides by automated solid-phase methods, using readily available reagents and equipment (see, for example, U.S. Pat. No. 4,458,066; or a review of the art in “Perspectives in Nucleoside and Nucleic Acid Chemistry”; ISBN: 3-90639-021-7, herein incorporated by reference). Alternatively, an H-phosphonate method of synthesis may be used

The completed modified oligonucleotide is then cleaved from the support and deprotected by treatment, e.g. with concentrated ammonium hydroxide, usually with a milder deprotection treatment using methods known in the art, e.g. potassium carbonate/methanol. A subsequent heat treatment removes the remaining protecting groups. The final product may purified by chromatography or electrophoresis, including ion exchange, HPLC, PAGE, etc. In some cases, crude oligonucleotides can be precipitated, or passed over a desalting column, and used without further purification.

### Methods of Use

Oligonucleotides comprising one or more of the subject analog nucleosides may be used in any of the techniques known in the art for such molecules, including use as probes in hybridization, as anti-sense probes for inhibition of expression; as RNAi for inhibition of expression; as a primer for PCR; a primer for RNA synthesis, and the like. In one aspect, the presence of polyfluorinated nucleosides stabilizes the oligonucleotide, and therefore provides a stabilized oligonucleotide composition, and a method for stabilization by introduction of the nucleoside analog into an oligonucleotide.

The properties conferred on an oligonucleotide by the presence of the analog nucleoside(s) also include an increase in hydrophobicity. The increased hydrophobicity provides for an advantage in the delivery of an oligonucleotide, particularly where delivery is across a cell membrane. Thus, in one embodiment of the invention; a method is provided for increasing the delivery of an oligonucleotide into a cell by providing for the presence of one or more polyfluorinated nucleosides in the oligonucleotide. Similarly, the increased hydrophobicity enhances the ability of an oligonucleotide to be presented in a lipid context, e.g. on the surface of a liposome or other lipid bilayers.

Increased hydrophobicity also provides for improvements in the isolation or purification of an oligonucleotide, e.g. by permitting the use of reverse phase HPLC in a purification procedure. A method is therefore provided for improved methods of isolation of an oligonucleotide by providing for the presence of one or more polyfluorinated nucleosides in the oligonucleotide.

Other methods of use rely on the selective base pairing conferred by the analog bases. The ability of the polyfluorinated analog bases to selectively self-pair, or pair with other polyfluorinated analog bases provides for a complementarity that is parallel to, but distinct from the pairing of native bases. By the inclusion of one, two or more polyfluorinated analog bases in an oligonucleotide sequence, the sequence can be designed to hybridize only with a similarly modified sequence, and not to an otherwise complementary native nucleic acid sequence. Thus, a method is provided for selective base pairing of single stranded sequences, by contacting two oligonucleotide sequences, in which polyfluorinated bases stably interact with each other, in preference to unmodified G, A, T, C, or U bases. Similarly, a stable double stranded helix is provided, comprising at least one pair of complementary polyfluorinated bases.

### Therapeutic Methods

The compounds of this invention can be incorporated into a variety of formulations for therapeutic administration. Of particular interest for therapeutic purposes are anti-sense oligonucleotides; double stranded RNAi oligonucleotides; and nucleosides. Certain nucleoside analogs are well known in the art for their ability to inhibit DNA and/or RNA replication. Such analogs have found use in the inhibition of viral replication (see, for example, U.S. Pat. Nos. 6,825,177; 6,818,633; 6,815,542; 6,809,109; 6,809,083; 6,803,371; 6,790,841, etc., herein incorporated by reference), e.g. in the treatment of herpesviruses; lentiviruses such as HIV-1; cytomegalovirus; etc. Such analogs have also found use in inhibiting the proliferation mammalian cells, including inflammatory cells such as T cells; and tumor cells. The polyfluorinated base analogs of the present invention may also be administered for such purposes.

For inhibition of gene expression, antisense oligonucleotides may be used in modulating the function of nucleic acid molecules encoding a polypeptide of interest (see, for example, U.S. Pat. Nos. 6,828,151; 6,828,149; 6,825,337; etc., herein incorporated by reference), by providing antisense compounds that specifically hybridize with the mRNA of interest. The specific hybridization of an oligonucleotide compound with its target nucleic acid interferes with the normal function of the target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. The targeting process also includes determination of a site or sites within this gene for the antisense interaction to occur such that the desired effect, e.g., detection or modulation of expression of the protein, will result. A preferred intragenic site is the region encompassing the translation initiation or termination codon of the open reading frame (ORF) of the gene. The open reading frame (ORF) is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap region may also be a preferred target region. Once one or more target sites have been identified, oligonucleotides are chosen which are sufficiently complementary to the target, i.e., hybridize sufficiently well and with sufficient specificity, to give the desired effect.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

For use in kits and diagnostics, the antisense compounds of the present invention, either alone or in combination with other antisense compounds or therapeutics, can be used as tools in differential and/or combinatorial analyses to elucidate expression patterns of a portion or the entire complement of genes expressed within cells and tissues. Expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

The specificity and sensitivity of antisense is also harnessed by those of skill in the art for therapeutic uses. Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

In pharmaceutical dosage forms, the compounds may be administered in the form of their pharmaceutically acceptable salts. They may also be used in appropriate association with other pharmaceutically active compounds.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host. Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

### Kits

Also provided are kits for practicing the subject methods. The kits according to the present invention may comprise at least: one modified oligonucleotide comprising one or more polyfluorinated analog bases; and (b) instructions for using the provided modified oligonucleotide(s). Such modified oligonucleotides may be provided lyophilized, in solution, or bound to a substrate. Kits may further include a second modified oligonucleotide to form a hybridizing pair.

Kits may also be provided for use in the synthesis of oligonucleotides comprising a polyfluorinated base analogs. Such kits may comprise modified H-phosphonate or phosphoroamidite derivatives to introduce polyfluorinated base analogs into a polynucleotide. The kits may further comprise additional reagents which are required for or convenient and/or desirable to include in the reaction mixture prepared during the subject methods, where such reagents include phosphoroamidite reagents and buffers for DNA synthesis; columns.

The various reagent components of the kits may be present in separate containers, or may all be precombined into a reagent mixture for combination with samples. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### Experimental

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLE 1

### Fluorinated DNA Bases as Probes of Electrostatic Effects in DNA Base Stacking

The noncovalent interactions affecting the thermodynamic stability of natural and modified DNA have been topics of broad interest in recent years. The effects of sterics, stacking, hydrogen bonding, and minor-groove salvation have been considered as contributing factors. Probably the dominant stabilizing factor in helical DNA is base stacking. In order to probe the physical factors that contribute to the stability of this stacking in water, measured melting data of short DNA oligomers, both naturally and nonnaturally substituted, has been studied. Such experiments have suggested that van der Waals and solvophobic forces can be important contributors to the stabilization of stacking. Beyond this, theoretical work has pointed out the possible importance of electrostatic interactions in the stability and preferred geometry of stacked bases in DNA. Understanding these issues could allow for better design of modified DNAs, but relatively little experimental information is available on such electrostatic factors.

It is well documented that aromatic rings can exhibit significant dipolar and quadrupolar electrostatic interactions in certain environments. Studies in nonpolar solvents using aromatic compounds containing various electron-donating and -withdrawing substituents have demonstrated significant electrostatic effects in stacking energetics and geometries. Although in aqueous systems the electrostatic dipole effects are greatly diminished, localized electrostatic effects are still believed to play a role in governing neighboring base-pair geometries in DNA. Studies with small-molecule model systems in water have suggested that electrostatic effects are relatively weak, and that dispersive effects are a major factor governing stacking stability. Hydrophobic effects in such model systems appear to play only a small role, although this issue has been debated.

Quadrupolar interactions have also been documented in specialized cases; for example, benzene is capable of electrostatic interactions with molecules containing a positive charge, a demonstrated by well-documented cation-π interactions, even in aqueous systems. By contrast, perfluorobenzene, with its opposite quadrupolar sign, can stack well (in low polarity environments) with electron-rich aromatic rings. A recent computational study also showed the coordination by water in interactions with benzene and perfluorobenzene. Although benzene-, 2,4-difluorobenzene-, 2,4,5-trifluorobenzene-, and pentafluorobenzene-substituted deoxyribonucleosides and 4-monofluorobenzene ribonucleoside have been previously described, no information on their relative stacking abilities is available, nor is there any data on their interactions with varied neighboring DNA bases.

We now describe a series of fluorinated aromatic nucleoside analogues having widely varied dipole and quadrupole moments. We have studied the stacking thermodynamics of these compounds in short synthetic DNA duplexes, with all four neighboring nucleobases. The results shed light on the importance and origins of electrostatic interactions in DNA base stacking, and reveal some previously unrecognized structural and electrostatic effects that are useful in molecular design.

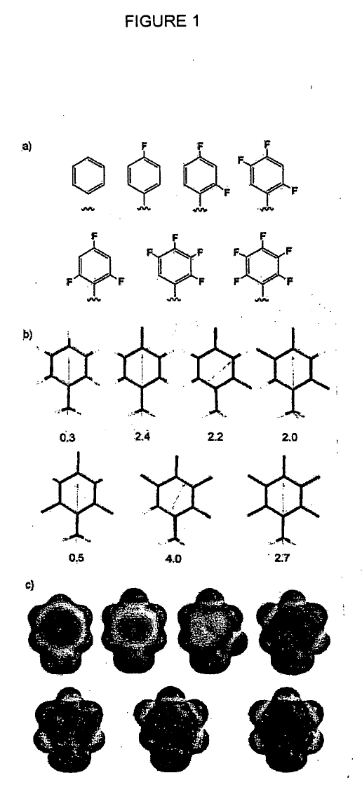

The seven deoxynucleosides studied here are shown in FIG. 1. In all cases, the deoxyribose moiety is constant, but the “base “groups vary in the extent and orientation of fluorine substitution, from zero substitutions (benzene) to the maximum of five (pentafluorobenzene). These compounds were prepared by treating the appropriate lithiated aromatic species with an O-protected deoxyribonolactone derivative.

FIG. 1b depicts calculated electrostatic surface potentials of the aromatic base analogues, in which the effects of the deoxyribose 1′ carbon are approximated with an attached methyl group. The electrostatics vary widely over the series, with the phenyl nucleoside showing a negative potential at the center of the flat aromatic face, and the pentafluorinated case having a strongly positive potential. Thus the quadrupoles are gradually inverted over this series. By comparison, natural DNA bases are not as strongly polarized (in the quadrupolar sense), and the potentials are generally close to neutral (similar to monofluorobenzene). In addition to differences in quadrupoles, this series has a broad range of dipole moments (FIG. 1c), ranging from a calculated 0.4 (for the phenyl case) to 4.5 debye (for the tetrafluorinated compound). Generally, the dipole directional orientations are quite similar over the series (FIG. 1c). By comparison, natural DNA bases have dipole moments that are generally large, but with orientations that vary widely. Finally, to test the effects of location of the fluorine substituents on the base, we studied two different trifluoro-substituted analogues: the 2,4,5-trifluorophenyl and 2,4,6-trifluorophenyl deoxyribonucleosides.

The seven nucleoside analogues were incorporated into synthetic oligonucleotides to study their stacking propensities with natural DNA bases as neighbors. They were incorporated by standard methods on an automated DNA synthesizer, and were characterized in DNA by NMR spectroscopy and by mass spectrometry.

The dangling-end experimental method was utilized to evaluate the ability of the fluorinated aromatic base analogues to stabilize DNA duplexes when substituted directly adjacent to the helix. The relative stabilizations were measured by comparison of the energetics for helix—coil melting transition of the “core “duplex (lacking any “dangling “nucleotide) to that containing the extra nucleotide. Five short self-complementary sequence contexts were used; the sequences were chosen because they had been shown previously to be well-behaved thermodynamically in the dangling-end configuration. Data from two of the contexts are shown in Table 1, and data for the remaining three show similar trends. By use of these five contexts we were able to compare stacking effects of the base analogues with all four neighboring DNA bases in the helix.

TABLE 1
Stacking of fluoroaromatic nucleotides as measured by thermal denaturation studies in two sequence
contexts^{[a]}

| | Tm | ΔTm | −ΔH | −ΔS | −ΔG₃₇ | −ΔG₃₇ | ΔΔG |
|---|---|---|---|---|---|---|---|
| Dangling residue | [° C.]^{[b]} | [° C.] | [kcal]^{[c]} | [eu]^{[c]} | [kcal]^{[c]} | [kcal]^{[d]} | stacking |
| XCGCGCG | | | | | | | |
| none (core duplex) | 41.4 | | 43.6 | 115 | 8.0 ± 0.1 | 8.1 ± 0.1 | |
| phenyl | 49.1 | 7.8 | 44.7 | 115 | 9.2 ± 0.1 | 9.4 ± 0.1 | 1.2 ± 0.1 |
| 4-fluorophenyl | 51.2 | 9.8 | 64.9 | 175 | 10.3 ± 0.3 | 9.9 ± 0.2 | 2.0 ± 0.2 |
| 2,4-difluorophenyl | 52.5 | 11.2 | 51.7 | 134 | 10.0 ± 0.1 | 10.2 ± 0.1 | |
| 2,4,5-trifluorophenyl | 52.2 | 10.8 | 55.0 | 145 | 10.1 ± 0.3 | 10.0 ± 0.1 | 2.0 ± 0.2 |
| 2,4,6-trifluorophenyl | 45.3 | 4.0 | 37.3 | 104 | 8.4 ± 0.2 | 8.6 ± 0.1 | 0.4 ± 0.1 |
| 2,3,4,5-tetrafluorophenyl | 53.9 | 12.6 | 53.8 | 93 | 10.3 ± 0.2 | 10.2 ± 0.2 | 2.2 ± 0.2 |
| pentafluorophenyl | 44.2 | 2.9 | 39.1 | 99 | 8.5 ± 0.2 | 8.7 ± 0.1 | 0.5 ± 0.2 |
| XACAGCTGT | | | | | | | |
| none (core duplex) | 40.0 | — | 44.8 | 119 | 7.9 ± 0.1 | 8.1 ± 0.1 | |
| phenyl | 44.4 | 4.4 | 57.6 | 157 | 8.9 ± 0.2 | 9.0 ± 0.1 | 1.0 ± 0.1 |
| 4-fluorophenyl | 48.7 | 8.7 | 66.6 | 183 | 9.9 ± 0.1 | 9.6 ± 0.1 | 1.8 ± 0.1 |
| 2,4-difluorophenyl | 47.8 | 7.8 | 62.4 | 170 | 9.7 ± 0.2 | 9.6 ± 0.1 | 1.7 ± 0.1 |
| 2,4,5-trifluorophenyl | 48.9 | 9.0 | 67.8 | 186 | 10.1 ± 0.2 | 9.7 ± 0.1 | 1.9 ± 0.2 |
| 2,4,6-trifluorophenyl | 47.8 | 7.9 | 53.6 | 143 | 9.3 ± 0.2 | 9.4 ± 0.2 | 1.3 ± 0.2 |
| 2,3,4,5-tetrafluorophenyl | 50.9 | 10.0 | 76.3 | 212 | 10.7 ± 0.2 | 9.8 ± 0.2 | 2.3 ± 0.2 |
| pentafluorophenyl | 47.3 | 7.0 | 55.2 | 148 | 9.3 ± 0.0 | 9.3 ± 0.2 | 1.3 ± 0.1 |

^{[a]}Free energy of stacking (ΔΔ G° is calculated as the difference between the free energies of the duplexes containing dangling residues from the energy of the core duplex.
^{[b]}Conditions: 1 M NaCl, 10 mM sodium phosphate pH 7.0; 5.0 μM DNA-strand concentration for the Tₘ value shown.
^{[c]}Thermodynamic values calculated from Van't Hoff plots.
^{[d]}Average free energy from fits to individual melting curves.

Thermodynamics were obtained both by curve fitting and by the van't Hoff method. All the duplexes appeared to behave in a two-state fashion and had well-shaped melting curves indicative of cooperative interactions of the dangling ends. All of the seven unnatural bases displayed significant stabilization of the duplexes relative to the core sequences (Table 1). The least stabilized is the case with a dangling pentafluorophenyl nucleotide on the (dCGCGCG)₂ core duplex, which gives an increase in Tₘ of only 2.9° C. and contributes −0.5 kcalmol⁻¹ of stability (“ΔΔG stacking “in Table 1). Almost as poorly stabilizing is the 2,4,6-trifluorinated case, which will be discussed below. The largest stabilizing interaction is observed with the 2,3,4,5-tetrafluorophenyl dangling nucleotide in that same sequence, with a Tₘ increase of 12.6° C. and a large stabilization of −2.2 kcalmol⁻¹ compared to the unsubstituted core sequence.

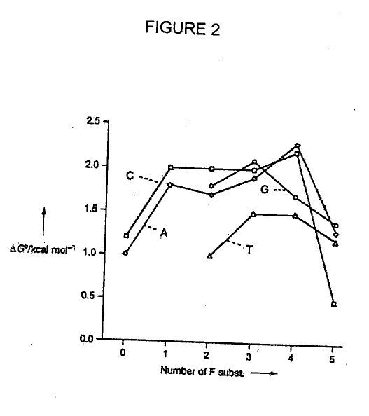

FIG. 2 shows the Tₘ data graphically, illustrating trends over the series with lines connecting data points. The “C adjacent “and “A adjacent “series have the most data available; here we see that mono-, di-,and trifluorinated bases (omitting, for the moment, the 2,4,6-trifluoro isomer) all stabilize the core sequences with similar propensity. For most-cases the previously unknown tetrafluoro analogue stacks somewhat more stably than the rest, while in all cases the previously known pentafluoro compound is surprisingly poor at stacking, behaving quite differently than the other compounds across the series.

It is worth noting that simple placement in a 5 ‘dangling position does not always guarantee a stacked orientation with the neighboring DNA bases. However, we expect that, since these analogues are relatively similar in size and geometry, their propensities for preferred geometries in DNA might also be similar. It will be important in the future to confirm geometries by structural studies, particularly in a strongly stabilizing case such as the tetrafluorinated analogue, as well as in a poorly stabilized (and potentially distorted) case such as with pentafluorobenzene. Regardless of geometry, however, the present results do indicate which structures are most stabilizing for future molecular designs.

We hypothesized that steric factors might contribute to the poor stabilization by the pentafluorinated species. Although fluorine is a relatively small substituent, it is generally accepted that even small groups can alter the glycosidic orientational preference in nucleosides, presumably by steric interactions with neighboring bonds and substituents. To test this further we prepared a second trifluorinated species, this one with 2,4,6 substitution, for direct comparison to the 2,4,5-substituted case. Measurement in the dangling-end contexts revealed that, like the pentafluorinated case, the bis-ortho-substituted trifluorobenzene case was very poor at stabilization (Table 1). This is a remarkable difference: a change of 6.8 8 (1.6 kcal) on moving one fluorine atom from the ortho to the meta position. Thus the data establish that bis-ortho substitution by even small fluorine groups can have a surprisingly large effect on stabilization, causing stacking of both the pentafluoro- and 2,4,6-trifluoronucleosides to be disrupted. We hypothesize that this may be due to a sterically induced twist in the glycosidic bond and/or in the sugar; structural studies will be helpful in evaluating this in the future. This finding explains the previously observed strong destabilizations seen in DNAs containing pentafluorobenzene.

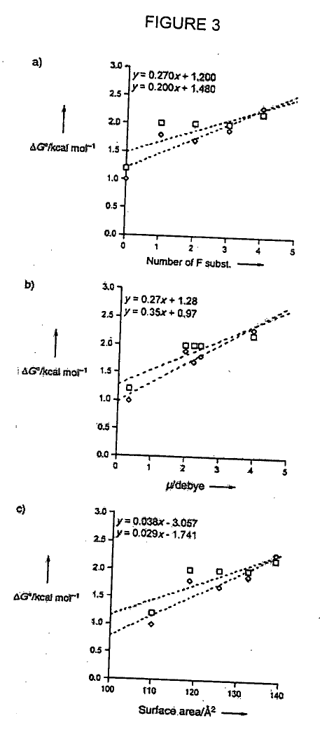

There is a gradual inversion of electrostatic potential at the centers of the flat aromatic surfaces going from the phenyl nucleoside to the pentafluorophenyl nucleoside across this series (see the electrostatic potential maps in FIG. 1b). It was anticipated that this difference in electrostatic potential might be a significant factor in the stacking of these compounds in duplex DNA, particularly with differing natural adjacent bases. Electrostatic maps of the four natural DNA bases suggest that adenine is the most electron-rich, while thymine is the least, although the differences are relatively small. However, from the Tₘ and ΔG° differences observed here (Table 1 and FIG. 2), there appears to be relatively poor correlation between numbers of fluorine groups and stabilization. For example, in the “C adjacent “and “A adjacent “series, the mono-, di-, and trisubstituted cases show nearly the same stacking propensities (FIG. 3a). (Note that we omit the two bis-ortho cases from the analysis because of their unusual steric effects.)

Since the tetrafluorinated species does stack somewhat more strongly, we cannot entirely rule out a quadrupolar effect, but the data suggest (see below) that simple dipole effects may provide a more consistent explanation for the results. Thus we conclude that, when at least one natural DNA base is involved, quadrupole effects are small or nonexistent, even with one strongly polarized partner. It remains to be seen, however, whether two adjacent non-natural stacking partners (which could be more strongly polarized than natural bases are) might exhibit quadrupolar stabilization or destabilization in water.

Dipole moments of the aromatic nucleobase analogues were calculated and plotted against Δ Tₘ and ΔΔA G° (stacking) for both the “C adjacent “and “A adjacent “series (FIG. 3b). Overall, it does appear that there is a linear correlation between dipole moments and stacking stabilization across these series. However, if permanent dipole effects are real, then the orientation of the analogue dipoles relative to the dipole orientations of adjacent bases should play a role in the electrostatic effects. Since the analogue dipole orientations are all similar (FIG. 1c), one should compare the dipole orientations of the neighboring bases, which differ more greatly. The dipole directions for adenine and cytosine are oriented roughly 45° relative to one another. If one assumes that a 5′ stacked base takes on the standard B-form conformation, then the dipole orientations of the analogues should be nearly opposed (180° to that of a cytosine in the neighboring helix, whereas they should be only partially opposed to that of a neighboring adenine. Also possibly significant is the much stronger dipole of C (6.0 debye as the 1-methyl derivative) relative to that of A (2.3 debye). These factors lead to the prediction of stronger dipole effects for a C neighbor than for an A neighbor. However, the plot (FIG. 3b) shows very similar slopes for the two series, which is not consistent with these predictions.

A dipole in an end-stacked nucleobase can have two types of electrostatic effects on stacking: a direct electrostatic interaction with a nearby permanent dipole, and also in the dispersive sense, by inducing an opposing dipole in the neighboring base. One would predict a stronger dispersive effect for a dangling base with a neighboring A than with a neighboring C, because of the greater polarizability of A. However, in the converse sense, C should induce a stronger dipole in the dangling base because of C's strong dipole. These opposing effects might tend to make C and A somewhat similar in stacking abilities, which is consistent with the similar slopes in FIG. 3b.

Thus we tentatively conclude that, while dipole effects appear to be significantly stabilizing to DNA base stacking, the origin of the effect may lie in their contribution to van der Waals dispersive forces. Overall, dipole effects can explain only roughly half (and likely less) of the stabilization observed here. Extrapolation to zero dipole still leaves about half of the stacking stabilization intact. Moreover, the compound with largest dipole (the tetrafluorobenzene case) also has greater surface area than the parent benzene compound, a factor that likely also contributes favorably. Plots of surface area vs. stacking (FIG. 3c) do show an apparent loose correlation of stabilization with surface area. A correlation of stacking with surface area has been reported previously. It remains to be seen whether the favorable effects of size in this case are due to solvophobic or dispersive effects.

Overall, our data are consistent with the notion that dispersive van der Waals attractions may be among the most important factors in DNA base stacking. The current results suggest that the electrostatic effects of nucleobase dipoles are significant in stabilizing stacking, but may explain only one-third to one-half of the stabilization for bases with strong dipoles. We further suggest that this dipole effect may be a result of dispersive induced-dipole attractions rather than of attractions between permanent dipoles. This leads to the suggestion that aromatic bases with large size and large dipole may be generally well-suited for stacking. Overall the data suggest that quadrupole interactions appear to be small for the natural bases, which have weak quadrupole moments. Finally, a previously unrecognized bis-ortho difluoro substitution effect was the largest factor observed in this series. This effect is clearly to be avoided in future designs of base analogues for helix stabilization.

Materials and Methods

Synthesis and Characterization of Nucleosides

General synthetic methods. All ¹³C and ¹H spectra were taken on a Varian Inova 500 spectrometer, a Varian XL 400 spectrometer, or a Varian Mercury 400 spectrometer. Chemical shifts are reported in ppm on the δ scale with the solvent given in the experimental for each compound as an internal reference. High resolution mass spectra were taken at the University of California at Riverside Mass Spectrometry Facility (UCRMS), Riverside, Calif. All flash chromatography was performed with Selecto Scientific 32-63 40UM Silica Gel.

Reactions were monitored by thin layer chromatography (TLC) on Silica Gel 60 (Merck) F-254 precoated 0.25 mm plates. Products were visualized by either UV light or staining with heated ceric ammonium sulfate (0.2% (w/v) cerium sulfate, 4.8% (w/v) ammonium molbdate and 10% (w/v) sulfuric acid) stain.

Dichloromethane (CH₂Cl₂), acetonitrile (CH₃CN) were dried by distillation from calcium hydride. Pyridine was dried by distillation from barium oxide. Tetrahydrofuran (THF) was distilled from sodium metal/benzophenone.

Synthesis. The previously described method of C-nucleoside coupling was utilized to generate the new fluorinated aromatic nucleosides 1-6′ as the tetraisopropyl disiloxane. (Scheme 1). The method involves the reaction of aryllithium derivatives of the fluorinated aromatic species with 3′,5′-O-((1,1,3,3-tetraisopropyl)disiloxanediyl)-2′-deoxy-D-ribono-1,4-lactone in hexanes at −78° C. to give a hemiketal which is then reduced to give the protected nucleoside. As seen in previous reactions with other aryllithium derivatives, these couplings give the β-anomer with high selectivity and comparable yields. Measured ratios of the β-anomer (by NMR integration) ranged from 87% of the monofluorophenyl derivative to >99% of the pentafluorophenyl derivative. Configuration at the C-1′ carbon was determined by ₁H NOE studies, as previously described. The siloxane was then deprotected using TBAF to give the free nucleoside in yields ranging from 50 to 85%. 2,4-difluorophenyl and 2,4,5-trifluorophenyl free nucleosides have been previously synthesized, but they have not previously been incorporated into oligonucleotides via synthetic means. 4-fluorophenyl ribonucleoside has been previously reported. However, the deoxyribonucleoside has not been previously synthesized. The phenyl free nucleoside has been previously reported. Others and we simultaneously synthesized the pentafluorophenyl free nucleoside. Standard methods were used to convert the free unprotected nucleosides to 5′-dimethoxytrityl-protected derivatives in yields ranging from 52 to 97%. These derivatives were then converted into cyanoethyl phosphoramidite derivatives, purified by column chromatography to give 38-82% yields.

Procedure for Lactone Coupling Reaction and Isolation of Protected Nucleoside. 1-Bromo-4-fluorobenzene (7.0 g, 40.0 mmol) was dissolved in hexane (99.5%+, 5 mL) and cooled to −78° C. t-Butyllithium (˜1.7M in pentane, 2.5 eq.) was added slowly to the mixture and stirred for 30 minutes under N₂ atmosphere. 3′,5′-O-((1,1,3,3-tetraisopropyl)disiloxanediyl)-2′-deoxy-D-ribono-1,4-lactone (3.0 g, 8.0 mmol) was dissolved in dry THF (5 mL) was added dropwise to the mixture and allowed to stir at −78° C. for 3 hours. The reaction was quenched with saturated aqueous NH₄Cl at −78° C. and allowed to warm to room temperature. The solution was extracted with ether and washed with saturated aqueous NH₄Cl, water, and brine. The organic layer was dried over MgSO₄ and concentrated as a yellow oil. Without purification, the mixture was dissolved in CH₂Cl₂ (6 mL) and cooled to −78° C. in an inert atmosphere. Triethylsilane (3 equivalents) and Boron trifluoride etherate (3 equivalents) were added to the mixture and allowed to stir for 6 hours. The reaction was quenched with saturated aqueous NaHC_{O3} and allowed to warm to room temperature. The solution was extracted with ether and washed with saturated aqueous NaHCO₃, water, and brine. The organic layer was dried over MgSO₄, concentrated as a yellow oil, and purified by flash silica gel chromatography, eluting with 20% ethyl acetate in hexanes. The major product was obtained as a clear oil.

1′,2′-dideoxy-β-1′-(4-fluorophenyl)-3′,5′-O-((1,1,3,3-tetraisopropyl)disiloxanediyl)-D-ribofuranose (1a, 19% total yield, 87% β-epimer): ¹H NMR (CDCl₃, ppm) δ 7.32 (2H, m), 7.03 (2H, m), 5.15 (1H, dd), 4.55 (1H, m), 4.05 (1H, m), 3.82 (1H, m), 3.68 (1H, m), 2.24 (1H, ddd), 1.94 (1H, m), 1.07 (28H, t) ¹³C-NMR (CDCl₃, ppm) δ 163.5, 161.1, 137.0, 127.8, 122.6, 115.3, 87.9, 79.6, 73.8, 63.4, 44.7, 16.8, 12.8.

1′,2′-dideoxy-β-1′-(2,4,6-trifluorophenyl)-3′,5′-O-((1,1,3,3-tetraisopropyl)disiloxanediyl)-D-ribofuranose (6a, 29% total yield, 92% β-epimer): ¹H NMR (CDCl₃, ppm) δ 6.66 (2H, m), 5.48 (1H, q), 4.64 (1H, m), 4.02 (1H, m), 3.81 (1H, m), 3.68 (1H, m), 2.34 (1H, m) 2.13 (1H, m), 1.03 (28H, m) ¹³C-NMR (CDCₗ3, ppm) δ 162.9, 100.86, 88.1, 73.77, 70.8, 62.9, 41.6, 25.8, 16.6, 12.5.

1′,2′-dideoxy-β-1′-(2,3,4,5-tetrafluorophenyl)-3′,5′-O-((1,1,3,3-tetraisopropyl)disiloxanediyl)-D-ribofuranose (4a, 27% total yield, 93% β-epimer): ¹H NMR (CDCl₃, ppm) δ 7.15 (1H, m), 5.33 (1H, dd), 4.57 (1H, m), 4.06 (1H, m), 3.82 (1H, m), 3.72 (1H, m), 2.36 (1H, m), 1.88 (1H, m), 1.06 (28H, t) ¹³C NMR (CDCl₃, ppm) δ 148.3, 145.8, 145.2, 142.7, 141.5, 139.2, 138.1, 127.6, 87.1, 73.7, 62.2, 42.6, 16.9, 12.8.

1′,2′-dideoxy-β-1′-(pentafluorophenyl)-3′,5′-O-((1,1,3,3-tetraisopropyl)disiloxanediyl)-D-ribofuranose (5a, 50% total yield, 97% β-epimer): ¹H NMR (CDCl₃, ppm) δ 5.44 (1H, t), 4.70 (1H, m), 4.09 (1H, dd), 3.91 (1H, m), 3.85 (1H, m), 2.46 (1H, m), 2.37 (1H, m), 1.12 (28H, m) ¹³C NMR (CDCl₃, ppm) δ 146.3, 144.4, 142.0, 140.0, 138.6, 136.6, 114.5, 86.1, 73.1, 69.3, 62.9, 39.8, 17.1, 13.0.

General Procedure for Deprotection of Disiloxanediyl-Protected Nucleoside. Disiloxane-protected nucleoside was dissolved in dry THF (10 mL) at room temperature. Tetrabutylammonium fluoride (1.0M in THF, 3 eq.) was added dropwise to the solution and allowed to stir for 3 hours. The reaction was quenched with 5% ammonium carbonate (6 mL) and extracted with ether. The organic layer was then washed with 5% ammonium carbonate and brine and concentrated to give a yellow oil. Silica gel flash chromatography ensued with ethyl acetate giving a white crystalline solid.

1′-(4-fluorophenyl)-2′-deoxy-D-ribose (1b, 82% yield): ¹H NMR (CDCl₃, ppm) δ 7.32 (2H, m), 7.04 (2H, t), 5.15 (1H, dd), 4.45 (1H, m), 4.02 (1H, m), 3.84 (1H, dd), 3.75 (1H, dd), 2.25 (1H, ddd), 2.02 (1H, m) ¹³C NMR (CDCl₃, ppm) δ 127.7, 115.4, 87.2, 79.5, 73.8, 63.4, 44.2 HRMS (DEI) calcd. for C₁₁H₁₃O₃F₁ (M+) 212.0849; found 212.0844.

1′,2′-dideoxy-β-1′-(2,4,6-trifluorophenyl)-D-ribofuranose (6b, 85% yield): ¹H NMR (CDCl₃, ppm) δ 6.66 (2H, t), 5.48 (1H, q), 4.51 (1H, m), 3.96 (1H, q), 3.80 (1H, dd), 3.72 (1H, dd), 2.40 (1H, m), 2.14 (1H, m) ¹³C NMR (CDCl₃, ppm) δ 163.4, 162.8, 161.4, 160.8, 112.2, 100.8, 87.2, 73.4, 70.6, 62.8, 40.8 HRMS (DCl) calcd. for C₁₁H₁₁O₅F₃ (M+NH₄+) 266.1004; found 266.0999.

1′,2′-dideoxy-β-1′-(2,3,4,5-tetrafluorophenyl)-D-ribofuranose (4b, 76% yield): ¹H NMR (CDCl₃, ppm) δ 7.14 (1H, m), 5.33 (1H, dd), 4.47 (1H, m),.4.03 (1H, m), 3.84 (1H, dd), 3.78 (1H, dd), 2.37 (1H, m), 1.96 (1H, m) ¹³C NMR (CDCl₃, ppm) δ 108.2, 87.1, 73.5, 63.2, 42.9 HRMS (DCl) calcd. for C₁₁H₁₀O₃F₄ (M+) 266.0566; found 266.0571.

1′,2′-dideoxy-β-1′-(pentafluorophenyl)-D-ribofuranose (5b, 50% yield): ¹H NMR (1:4 CD₃OD:CDCl₃, ppm) δ 5.44 (1H, dd), 4.35 (1H, m), 4.11 (2H, d), 3.89 (1H, m), 3.63 (2H, m), 2.33 (1H, m), 2.15 (1H, m) ¹³C NMR (1:4 CD₃OD:CDCl₃, ppm) δ 146.4, 143.9, 141.7, 139.3, 138.6, 136.1, 113.7, 87.5, 72.5, 70.3, 62.4, 39.6 HRMS (DCl+NH₄+) calcd. for C₁₁H₁₉O₃F₅ (M+) 302.0816; found 302.0827.

General Procedure for Preparation of 5′-O-tritylated β-C-Nucleosides. The above-synthesized nucleoside (0.08 g, 0.40 mmol)) was dissolved in a 1:1 mixture of pyridine and methylene chloride (10 mL). Diiospropylethylamine (0.10 mL, 0.60 mmol) and 4,4′-dimethoxytrityl (DMT) chloride (0.26 g, 0.80 mmol) were added to mixture and stirred for 4 hours at room temperature and then quenched with methanol (8 mL). The resulting mixture was concentrated and purified by flash chromatography, eluting with 20% ethyl acetate in hexanes. The product was concentrated as a yellow foam, 1′,2′-dideoxy-â-1′-(2,4-difluorophenyl)-5′-O-trityl-D-ribofuranose (2c, 86% yield): ¹H NMR (CDCl₃, ppm) δ 7.50 (1H, q), 7.46 (2H, m), 7.34 (4H, d), 7.27 (2H, t), 7.22 (1H, t), 6.83 (4H, d), 6.80, (1H, m), 6.77 (1H, m), 5.38 (1H, q), 4.42 (1H, m), 4.05 (1H, m), 3.80 (6H, s), 3.36 (1H, dd), 3.28 (1H, dd), 2.32 (1H, m), 1.99 (1H, m) ¹³C NMR (CDCl₃, ppm) δ 163.1, 161.2, 160.6, 158.5, 144.7, 135.9, 130.1, 128.1, 127.7, 126.8, 113.1, 86.2, 74.5, 73.6, 64.2, 55.2, 42.5 HRMS (FAB, 3-NBA matrix) calcd. for _{C3}2_{H3}0_{O}5_{F2} (M+Na) 555.1959; found 555.1975.

1′,2′-dideoxy-β-1′-(2,4,5-trifluorophenyl)-5′-O-trityl-D-ribofuranose (3c, 97% yield): ¹H NMR (CDCl₃, ppm) δ 7.44 (2H, d), 7.24 (9H, m), 6.84 (4H, d), 5.32 (1H, q), 4.44 (1H, m), 4.05 (1H, m), 3.79 (6H, s), 3.35 (1H, dd), 3.26 (1H, dd), 2.35 (1H, m), 1.96 (1H, m), ¹³C NMR (CDCl₃, ppm) δ 158.5, 144.8, 135.8, 130.0, 129.1, 128.1, 127.7, 127.1, 113.1, 86.0, 74.3, 73.6, 64.2, 55.2, 42.9 HRMS (FAB, 3-NBA matrix) calcd. for C₃₂H₂₉O₅F₃ (M+Na) 573.1865; found 573.1886.

1′,2′-dideoxy-β-1′-(2,4,6-trifluorophenyl)-5′-O-trityl-D-ribofuranose (6c, 52% yield): ¹H NMR (CDCl₃, ppm) δ 7.45 (2H, d), 7.34 (4H, d), 7.27 (2H, t), 7.20 (1H, t), 6.81 (4H, d), 6.62 (2H, t), 5.44 (1H, q), 4.45 (1H, m), 3.95 (1H, m), 3.77 (6H, s), 3.38 (1H, m), 3.21 (1H, m), 2.40 (1H, m), 2.09 (1H, m) ¹³C NMR (CDCl₃, ppm) δ 171.3, 163.3, 162.8, 161.3, 160.8, 158.4, 144.8, 136.0, 130.0, 128.1, 127.8, 126.7, 113.0, 86.2, 74.4, 70.1, 64.2, 55.1, 45.0, 39.5 HRMS (FAB, 3-NBA matrix) calcd. for C₃₂H₂₉O₅F₃ (M+) 550.1967; found 550.1986.

1′,2′-dideoxy-β-1′-(2,3,4,5-tetrafluorophenyl)-5′-O-trityl-D-ribofuranose (4c, 91% yield): ¹H NMR (CDCl₃, ppm) δ 7.43 (2H, d), 7.33 (4H, dd), 7.29 (2H, t), 7.22 (1H, m), 6.83 (4H, d), 5.35 (1H, q), 4.41 (1H, m), 4.08 (1H, m), 3.77 (6H, s), 3.33 (1H, q), 3.25 (1H, q), 2.37 (1H, m), 1.93 (1H, m), 1.26 (1H, s) ¹³C NMR (CDCl₃, ppm) δ 158.5, 149.4, 148.0, 146.1, 145.2, 144.6, 143.2, 141.3, 140.3, 139.3, 138.3, 136.3, 135.7, 130.0, 127.9, 126.9, 126.2, 123.9, 113.1, 86.2, 74.0, 73.0, 64.1, 55.1, 42.5 HRMS (FAB, 3-NBA matrix) calcd. for C₃₂H₂₈O₅F₄ (M+) 568.1873; found 568.1897.

1′,2′-dideoxy-β-1′-(pentafluorophenyl)-5′-O-trityl-D-ribofuranose (5c, 78% yield): ¹H NMR (CDCl₃, ppm) δ 7.43 (2H, d), 7.34 (4H, d), 7.27 (2H, m), 7.22 (1H, m), 6.82 (4H, m) 5.44 (1H, q), 4.44 (1H, m), 3.96 (1H, m), 3.78 (6H, s), 3.39 (1H, dd), 3.20 (1H, dd), 2.35 (1H, m), 2.16 (1H, m) ¹³C NMR (CDCl₃, ppm) δ 158.47, 144.70, 135.86, 130.02, 128.08, 127.83, 126.82, 113.10, 86.34, 86.11, 74.20, 70.25, 64.38, 63.98, 55.18, 39.67, 30.60, 19.09, 13.69 ₁₉F-NMR (CDCₗ3, ppm) ä-142.51 (2F, m), −154.93 (1F, t), −162.65 (2F, m) HRMS (FAB, 3-NBA matrix) calcd. for C₃₂H₂₇O₅F₅ (M+) 586.1779; found 586.1797.

General Procedure for Preparation of 3′-O-Phosphoramidites. The 5′-O-tritylated nucleoside (0.17 g, 0.33 mmol) was dissolved in dry methylene chloride (5 mL) under N₂ atmosphere. Diisopropylethylamine (0.25 mL, 1.4 mmol) and 2-cyanoethyl N,N-diisopropylchlorophosphoramidite (0.50 mL, 2.2 mmol) were added to the mixture and stirred for 4 hours at room temperature. The mixture was concentrated and purified by flash silica gel column chromatography, eluted with 3% triethylamine/20% ethyl acetate in hexanes. The product was obtained as an oil.

1′,2′-dideoxy-β-1′-(4-fluorophenyl)-5′-O-trityl-D-ribofuranose phosphoramidite (1d, 43% yield): ¹H NMR (CDCl₃, ppm) δ 7.48 (2H, m), 7.36 (6H, m), 7.24 (3H, m), 7.02 (2H, m), 6.82 (4H, m), 5.14 (1H, q), 4.52 (1H, m), 4.22 (1H, m), 3.69-3.87 (2H, m), 3.79 (6H, s) 3.59 (2H, m), 3.27 (2H, m), 2.62 (1H, t), 2.01 (1H, m), 1.18 (10H, m), 1.08 (4H, d) ¹³C NMR (CDCl₃, ppm), δ 158.4, 144.8, 136.0, 130.1, 129.1, 128.2, 127.8, 126.7, 115.1, 113.1, 86.1, 79.7, 64.1, 58.3, 55.2, 43.2, 24.5, 20.3 HRMS (FAB, 3-NBA matrix) calcd. for C₄₁H₄₈N₂O₆F₁P (M+Na) 737.3132; found 737.3166.

1′,2′-dideoxy-β-1′-(2,4-difluorophenyl)-5′-O-trityl-D-ribofuranose phosphoramidite (2d, 72% yield): ¹H NMR (CDCl₃, ppm) δ 7.55 (1H, m), 7.46 (2H, m), 7.35 (4H, m), 7.28 (2H, m), 7.21 (1H, m), 6.82 (6H, m), 5.35 (1H, q), 4.51 (1H, m), 4.21 (1H, m), 3.82 (3H, m) 3.79 (6H, s), 3.69 (1H, m), 3.60 (2H, m), 3.35 (1H, dd), 3.27 (1H, dd), 2.62 (1H, t), 2.45 (1H, m), 1.18 (12H, m) ¹³C NMR (CDCl₃, ppm) δ 158.4, 144.8, 136.0, 130.1, 128.2, 127.8, 126.8, 113.0, 86.1, 75.7, 73.9, 63.9, 58.3, 55.2, 43.1, 41.9, 24.4, 20.2 HRMS (FAB, 3-NBA matrix) calcd. for C₄₁H₄₇N₂O₆F₂P (M+Na) 755.3038; found 755.3051.

1′,2′-dideoxy-â-1′-(2,4,5-trifluorophenyl)-5′-O-trityl-D-ribofiranose phosphoramidite (3d, 63% yield): ¹H NMR (CDCl₃, ppm) δ 7.46 (3H, m), 7.35 (4H, m), 7.28 (2H, m), 7.22 (1H, m), 6.90 (1H, m), 6.83 (4H, m), 5.32 (1H, m), 4.50 (1H, m), 4.24 (1H, m), 3.80 (3H, m) 3.79 (6H, s), 3.70 (1H, m), 3.59 (2H, m), 3.28 (2H, m), 2.62 (1H, t), 2.47 (1H, m), 1.23 (2H, m) 1.18 (10H, m) ¹³C NMR (CDCl₃, ppm) δ 158.5, 144.7, 135.8, 130.1, 128.2, 127.8, 126.8, 113.1, 86.1, 85.7, 85.4, 75.7, 73.5, 64.0, 58.3, 55.2, 43.1, 41.9, 39.1, 24.5, 20.2 HRMS (FAB, 3-NBA matrix) calcd. for C₄₁H₄₆N₂O₆F₃P (M+Na) 773.2943; found 773.2933.

1′,2′-dideoxy-β-1′-(2,4,6-trifluorophenyl)-5′-O-trityl-D-ribofuranose phosphoramidite (6d, 38% yield): ¹H NMR (CDCl₃, ppm) δ ₁H NMR (CDCl_{3, ppm) δ} 7.48 (2H, m), 7.37 (4H, m), 7.26 (2H, m), 7.18 (1H, m), 6.81 (4H, m), 6.63 (2H, m), 5.44 (1H, m), 4.57 (1H, m) 4.16 (1H, m), 3.76 (6H, s), 3.63 (3H, m), 3.28 (1H, m), 2.56 (1H, t), 2.43 (1H, m), 2.23 (1H, ddd), 2.03 (1H, d), 1.19 (14H, m) ¹³C NMR (CDCl₃, ppm) δ 162.8, 160.8, 158.2, 144.8, 135.9, 130.0, 130.0, 128.1, 127.6, 126.5, 117.4, 112.2, 100.4, 85.6, 75.4, 70.3, 63.5, 58.3, 58.1, 55.0, 43.1, 38.8, 24.3, 20.0 HRMS (FAB, 3-NBA matrix) calcd. for C₄₁H₄₆N₂O₆F₃P (M+Na) 773.2943; found 773.2929.

1′,2′-dideoxy-β-1′-(2,3,4,5-tetrafluorophenyl)-5′-O-trityl-D-ribofuranose phosphoramidite (4d, 76% yield): ¹H NMR (CDCl₃, ppm) δ 7.44 (2H, d), 7.33 (4H, m), 7.27 (3H, m), 7.17 (1H, m), 6.83 (4H, m), 5.34 (1H, m), 4.51 (1H, m), 4.23 (1H, m), 3.79 (6H, s), 3.77 (2H, m), 3.62 (2H, m), 3.29 (1H, t), 3.26 (1H, d), 2.64 (2H, m), 2.46 (1H, m), 1.91 (1H, m), 1.60 (1H, s), 1.19 (12H, m) ¹³C NMR (CDCl₃, ppm) δ 158.5, 144.7, 135.7, 132.2, 130.1, 129.1, 128.1,.127.8, 126.9, 117.4, 113.1, 108.3, 86.2, 75.6, 73.3, 70.6, 63.9, 58.3, 55.2, 43.2, 41.8, 24.6, 20.3 HRMS (FAB, 3-NBA matrix) calcd. for C₄₁H₄₅N₂O₆F4P (M+Na) 791.2849; found 791.2895.

1′,2′-dideoxy-β-1′-(pentafluorophenyl)-5′-O-trityl-D-ribofuranose phosphoramidite (5d, 82% yield): ¹H NMR (CDCl₃, ppm) δ 7.46 (2H, d), 7.33 (4H, m), 7.26 (2H, m), 7.20 (1H, m), 6.81 (4H, m), 5.43 (1H, q), 4.51 (1H, m), 4.16 (1H, q), 3.78 (6H, s), 3.75 (2H, m) 3.57 (2H, m), 3.27 (1H, q), 3.19 (1H, q), 2.40 (1H, m), 2.32 (1H, m), 1.56 (2H,s), 1.23 (12H, m) ¹³C NMR (CDCl₃, ppm) δ 158.4, 144.8, 136.0, 130.1, 128.2, 127.7, 126.7, 117.6, 113.0, 86.1, 75.2, 70.6, 63.5, 58.3, 55.2, 43.0, 39.1, 24.5, 20.3 HRMS (FAB, 3-NBA matrix) calcd. for C₄₁H₄₄N₂O₆F₅P (M+Na) 809.2755; found 809.2720.

0a, 0b, 0c, 0d, 2a, 3a, 2b, and 3b were synthesized as previously reported.

Synthesis and characterization of oligonucleotides. Oligonucleotide synthesis. The β-C-deoxynucleosides 0-6 were incorporated into DNA oligonucleotides by automated solid-phase methods. Oligonucleotides were synthesized using an Applied Biosystems 392 DNA/RNA synthesizer in trityl-off mode using standard β-cyanoethylphosphoramidite chemistry. Lengthened coupling times were used, giving stepwise yields of >95% by trityl monitoring. After synthesis, oligonucleotides were deprotected and removed from solid support in the usual manner. Oligomers were purified by HPLC and quantitated by UV absorption. Molar extinction coefficients were calculated by the nearest neighbor method. Values for oligonucleotides containing nonnatural residues were calculated by adding the extinction coefficient of the nonnatural nucleoside to the extinction coefficient of the core duplex. Intact structures of the oligonucleotides were confirmed by MALDI-TOF mass spectrometry.

Methods for thermodynamic measurements. Thermal denaturation. Solutions for thermal denaturation studies were prepared as 1 mL samples ranging between 1 μM and 40 μM concentrations in melt buffer (1 M NaCl, 10 mM sodium phosphate, pH=7.0). Solutions were then heated to 90° C. for 5 minutes and annealed by slowly cooling to room temperature and then to 0° C. The melting studies were carried out in Teflon-stoppered 1 cm pathlength quartz cells under nitrogen atmosphere on a Varian Cary 1 UV-Vis spectrophotometer equipped with a Peltier temperature controller. Absorbance was monitored at 280 nm while temperature was raised from 5 to 90° C. at a rate of 0.5° C./minute. In all cases, the complexes displayed sharp, apparently two-state transitions.

Calculations. Melting temperature (Tm) were determined by computer fit (Meltwin 3.5) of the first derivative of absorbance with respect to 1/T. Uncertainty of Tm is estimated at ±0.5° C. based on repetitions of experiments. Free energy values were derived by two methods: (1) computer-fitting the denaturation data with an algorithm employing linear sloping baselines, using the two-state approximation for melting and (2) van't Hoff thermodynamic parameters derived from linear plots of 1/Tm vs. ln(cT) by measuring Tm at varied concentration.

TABLE 2
Table S1. Stacking of Fluoroaromatic Nucleotides as Measured by
Thermal Denaturation Studies in Three Additional Sequence
Contexts ^{a}

| Dangling | Tₘ | Tₘ | −H | −S | −G₃₇ | −G₃₇ | G |
|---|---|---|---|---|---|---|---|
| Residue | (° C.) ^{b} | (° C.) | (kcal) ^{c} | (eu) ^{c} | (kcal) ^{c} | (kcal) ^{d} | stacking |
| XGTAGCTAC | | | | | | | |
| none (core duplex) | 34.4 | — | 53.9 | 151 | 7.1 0.0 | 7.0 0.1 | |
| 2,4-difluorophenyl | 43.4 | 9.0 | 62.7 | 173 | 8.9 0.1 | 8.8 0.2 | 1.8 0.1 |
| 2,4,5-trifluorophenyl | 44.4 | 10.1 | 72.5 | 204 | 9.3 0.1 | 9.1 0.3 | 2.1 0.2 |
| 2,4,6-trifluorophenyl | 42.0 | 7.6 | 52.5 | 142 | 8.4 0.1 | 8.5 0.0 | 1.3 0.0 |
| 2,3,4,5- | 43.5 | 9.1 | 57.0 | 156 | 8.8 0.1 | 8.8 0.1 | 1.7 0.1 |
| tetrafluorophenyl | | | | | | | |
| pentafluorophenyl | 42.3 | 7.9 | 56.5 | 155 | 8.4 0.0 | 8.4 0.0 | 1.4 0.0 |
| XTGAGCTCA | | | | | | | |
| none (core duplex) | 38.0 | — | 44.0 | 117 | 7.6 0.1 | 7.6 0.1 | |
| 2,4-difluorophenyl | 43.7 | 5.7 | 43.9 | 114 | 8.5 0.1 | 8.7 0.2 | 1.0 0.1 |
| 2,4,5-trifluorophenyl | 46.8 | 8.8 | 53.8 | 144 | 9.2 0.1 | 9.1 0.2 | 1.5 0.1 |
| 2,4,6-trifluorophenyl | 45.5 | 7.5 | 46.7 | 122 | 8.8 0.0 | 9.0 0.2 | 1.3 0.1 |
| 2,3,4,5- | 46.5 | 8.5 | 51.9 | 138 | 9.1 0.1 | 9.2 0.1 | 1.5 0.1 |
| tetrafluorophenyl | | | | | | | |
| pentafluorophenyl | 45.2 | 7.2 | 48.6 | 128 | 8.7 0.1 | 8.9 0.1 | 1.2 0.1 |
| XAGCGCT | | | | | | | |
| none (core duplex) | 33.1 | — | 48.6 | 134 | 6.9 0.0 | 7.0 0.1 | |
| 2,3,4,5- | 42.7 | 9.6 | 48.4 | 129 | 8.3 0.0 | 8.3 0.0 | 1.4 0.0 |
| tetrafluorophenyl | | | | | | | |
| pentafluorophenyl | 39.9 | 6.8 | 45.0 | 120 | 7.9 0.0 | 7.8 02 | 0.9 0.1 |

### EXAMPLE 2

### Selective Pairing of Polyfluorinated DNA Bases

Recent studies of polyfluorinated organic compounds have revealed useful selective interactions between such “fluorous” species relative to their interactions with water or the parent hydrocarbons. Although the origins of this selective interaction are not fully understood, it has been established that perfluorinated hydrocarbons are significantly more hydrophobic than the analogous hydrocarbons. The shielding of strongly hydrophobic surfaces from solvent may explain selective polyfluorinated side-chain pairing, recently employed successfully in peptide-peptide interactions, and in enhanced stability of proteins with fluorinated amino acids. Fluorocarbon interactions have been gaining widespread utility and interest, especially in aiding separations of catalysts, reagents, substrates, and products.

We sought to establish whether such fluorocarbon selectivity could be harnessed in pairing of DNA bases, as an alternative to other known modes of pairing. A number of laboratories have investigated molecular strategies for selective base pairing, using approaches other than that of nature itself, namely Watson-Crick hydrogen bonding. Such work has yielded fundamental information on DNA biophysics and biology, and applied utility in expansion of the genetic alphabet. Benner proposed over a decade ago that hydrogen-bonding schemes beyond the standard Watson-Crick arrangement could be used in selective pairing. In more recent years, strategies that avoid hydrogen bonding altogether have been introduced, employing selective pairing of hydrocarbon “bases” with one another. Such selectivity can arise from the avoidance of the energetic cost of desolvation of polar natural bases and from the advantageous burying of hydrophobic surface area.

Although nucleic acid base analogues with fluorine substituents have been reported by Schweitzer et al. (1994) J. Org. Chem. 1994, 59, 7238-7242; Parsch et al. (2000) Chim. Acta 2000, 83, 1791; Shibata et al. (2001) J. Chem. Soc., Perkin Trans. 14, 1605-1611; Mathis et al. (2002) Angew. Chem., Int. Ed. 41, 3203-3205; and Lai et al. (2003) Angew. Chem., Int. Ed. 42, 5973-5977; there is as yet no report on whether highly fluorinated DNA bases might display a selective interaction with one another, analogous to the pairing of polyfluorinated peptides. A recent report described pentafluorobenzene as a DNA base replacement, but no selective pairing was observed in oligonucleotides. Subsequent studies have revealed, however, that pentafluorobenzene is strongly destabilizing to helical DNA because of an unfavorable effect of two ortho fluorine substituents. This suggested that selective pairing might still be possible if other highly fluorinated DNA base analogues were to be examined, as long as this bis-ortho effect were avoided.

Examination of DNA models suggested a number of polyfluorinated DNA base replacements as possible candidates for selective fluorous pairing. The modeling suggested that 2,3,4,5-tetrafluorobenzene (abbreviated ^{F}B) might pair opposite itself without distorting the helical geometry. Moreover, this compound has recently been shown to stack quite strongly at the ends of DNA helices. A second candidate was the previously unknown 4,5,6,7-tetrafluoroindole (^{F}I), which was readily synthesized as an N-nucleoside species. These two deoxyribosides were prepared as their 5′-trityl,3′-phosphoramidite derivatives for incorporation into DNA by automated synthesizer.

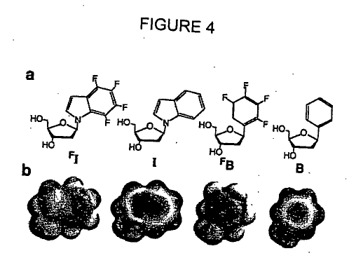

For comparison we also prepared the non-fluorinated hydrocarbon analogues, phenyl (B) and indole (I) glycoside derivatives (FIG. 4). To test pairing preferences of the four unnatural base replacements, we placed them in short oligonucleotides and paired these strands with complementary partners containing either natural bases or unnatural analogues at single or double positions. Stabilities of the duplexes were evaluated by thermal denaturation monitored by UV absorbance, in a pH 7.0 buffer containing 1.0 M NaCI, 10 mM Na phosphate, and 0.1 mM EDTA. Melting temperatures were determined from the inflection points in the curves, and free energies were obtained by van't Hoff plots of the data at multiple DNA concentrations.

Initial experiments pairing the two fluorinated nucleosides opposite natural DNA bases in a 12-bp duplex confirmed they pair with low stability opposite the hydrophilic nucleobases. However, when paired opposite themselves, a significant degree of stability was regained for both compounds. This confirms that the pairing of the polyfluorinated bases operates selectively in the context of natural DNA, thus displaying significant levels of orthogonality. The ^{F}I-^{F}I pair in this context is nearly as stable as the natural T-A pair. The mild-to-moderate destabilization of the duplex by these pairs (as compared to natural base pairs) is consistent with several previous nonpolar DNA base-pair analogues and is most likely due to the energetic cost of desolvation.

We then began more detailed studies with a new sequence, increasing the nonpolar pair content to 2/12 (17%) to emphasize differences among various combinations of the nonnatural bases. The data confirm that all the self-complementary sequences give the expected concentration dependence, confirming two-stranded duplexes (as opposed to self-folded hairpins. Overall, the results show (Table 1, FIG. 2) that the fluorinated bases pair selectively with each other, as compared to the hydrocarbon-hydrocarbon pairing or the mixed fluorocarbon-hydrocarbon pairing. For example, the tetrafluorobenzene-tetrafluoroindole pair (^{F}B-^{F}I) is more stable than the similar benzene-indole pair lacking fluorine; the difference is a significant 14° C. in Tm and 1.5 kcal/mol in free energy.

The mixed versions of these pairs, placing hydrocarbon opposite fluorocarbon, resulted in pairing stabilities falling between those of the fully fluorinated and nonfluorinated pairs. The two other cases also confirm the selective pairing effect: the ^{F}B-^{F}B pair is more stable than the all-hydrocarbon B-B pair, and ^{F}I-^{F}I is more stable than I-I. The fluorinated ^{F}I-^{F}I pair is the most stable of the series, while the hydrocarbon B-B pair is the least stable. The difference between these extremes is 15.4° C. (2.1 kcal/mol), illustrating the significant degree by which structure and polyfluorination can affect base-pair stability.

To seek evidence for the origins of this selectivity, we examined individual properties of the four nonpolar nucleoside analogues. Stacking was evaluated by the standard dangling end approach using a 6-bp self-complementary DNA. Results showed that the two polyfluorinated analogues do, in fact, stack more strongly than the two parent hydrocarbons. ^{F}B stacks 1.0 kcal/mol more favorably than B, and ^{F}I, 1.1 kcal/mol more favorably than I.

We explicitly examined hydrophobicity of the four nucleosides by partitioning between 1-octanol and water. The data are as follows: B (log P) 0.77 (0.10); ^{F}B (1.39 (0.10); I (0.99 (0.10); ^{F}I (1.66 (0.10). The results confirm what has been previously reported for polyfluorinated saturated hydrocarbons: namely, that they are more hydrophobic than their hydrocarbon variants. For the present compounds, the order of hydrophobicity is ^{F}I>^{F}B>I>B. This correlates well with the stabilities of their self-pairs as well as with their relative stacking abilities.

Taken together, the data suggest that this selective pairing may be due to solvent avoidance of these specially hydrophobic structures on formation of a duplex relative to the more exposed single strands. Placing them in pairs opposite one another buries large fractions of the flat π surfaces and significant parts of the edges facing one another as well. Thus, the basic physical origins of the selective interaction appear to be similar to those seen recently in selective fluorinated peptide interactions.

Our findings demonstrate that polyfluoroaromatic base pairing can employed as a new, selective approach to pairing in DNA that is orthogonal to that of the natural genetic system. Future structural studies could shed light on the orientations of the base analogues in DNA. Also of interest is whether such fluorocarbon pairing selectivity could exert significant effects in the enzymatic replication of DNA.

TABLE 3
Thermodynamic Data for Duplexes Containing Fluorous
and Hydrocarbon Bases^{a}

| base pair^{b} | Tₘ^{c} | ¢ G°₃₇^{d} | ¢¢ G°₃₇^{e} |
|---|---|---|---|
| (X¥Y) | (° C.) | (kcal/mol) | (kcal/mol) |
| B.B | 29.8 | −6.7 ( 0.2 | −1.0 ( 0.4 |
| FB.FB | 34.6 | −7.3 ( 0.1 | −1.6 ( 0.3 |
| I.I | 31.5 | −7.2 ( 0.2 | −1.5 ( 0.4 |
| FI.FI | 45.2 | −8.8 ( 0.1 | −3.1 ( 0.3 |
| B.I | 27.8 | −6.7 ( 0.1 | −1.0 ( 0.3 |
| FB.FI | 41.8 | −8.2 ( 0.1 | −2.5 ( 0.3 |
| T.A | 58.1 | −11.9 ( 0.3 | −6.2 ( 0.5 |
| T.C | 20.3 | −5.7 ( 0.2 | — |

^{a}Conditions: 1 M NaCl, 10 mM phosphage (pH 7.0) with 0.1 mM EDTA.
^{b}Sequence is 5′-CGGXAGCTYCCG (self-complementary).
^{c}Tₘ values are at 5.0 1 M.
^{d}Averages of values from van't Hoff and curve fitting methods.
^{e}Values resolve to the least stable duplex (the T-C mismatch).

Materials and Methods

General synthetic methods. All ¹³C and ¹H spectra were taken on a Varian Inova 500 spectrometer, a Varian XL 400 spectrometer, or a Varian Mercury 400 spectrometer. Chemical shifts are reported in ppm on the δ scale with the solvent given in the experimental for each compound as an internal reference. High resolution mass spectra were taken at the University of California at Riverside Mass Spectrometry Facility (UCRMS), Riverside, Calif. All flash chromatography was performed with Selecto Scientific 32-63 40UM Silica Gel. Reactions were monitored by thin layer chromatography (TLC) on Silica Gel 60 (Merck) F-254 precoated 0.25 mm plates. Products were visualized by either UV light or staining with heated ceric ammonium sulfate (0.2% (w/v) cerium sulfate, 4.8% (w/v) ammonium molbdate and 10% (w/v) sulfuric acid) stain. Dichloromethane (CH₂Cl₂), acetonitrile (CH₃CN) were dried by distillation from calcium hydride. Pyridine was dried by distillation from barium oxide. Tetrahydrofuran (THF) was distilled from sodium metal/benzophenone.

Synthesis. The syntheses of the phenyl nucleoside ₁ and tetrafluorophenyl ₂ nucleoside have been previously described. N-nucleosides were synthesized by coupling the indole compound to 1′-chloro-2′-deoxy-3′,5′-di-O-p-toluoyl-a-D-erythropentofuranose in the presence of sodium hydride. (Scheme 2) Configuration at the C-1′ carbon was determined by ₁H NOE studies, as previously described. ₁ Measured ratios of the b-anomer (by NMR integration) for the tetrafluoroindole and indole nucleosides were 95% and 92%, respectively. Deprotection of toluoyl groups were performed using sodium methoxide. (Scheme 2) Standard methods were used to convert the free unprotected nucleosides to 5′-dimethoxytrityl-protected derivatives in yields ranging from 67 to 95%. These derivatives were then converted into cyanoethyl phosphoramidite derivatives, purified by column chromatography to give 67-81% yields.

General procedure for sugar coupling reaction and isolation of protected nucleoside. Tetrafluoroindole (1.0 g, 5.3 mmol) was dissolved in dry acetonitrile (20 mL) in a flame dried flask under _{N2} atmosphere. The solution was cooled to 0° C. Sodium hydride powder (192 mg, 8.0 mmol) was added in one amount to produce a cloudy suspension and stirred for 30 minutes. The temperature was cooled to −5° C. with a brine-ice bath and 1-chloro-2-deoxy-3,5-di-O-p-toluoyl-a-D-erthro-pentafuranose (2.1 g, 5.3 mmol) was added as a solid in a single portion. The mixture turned creamy brown. After 1 hour, the reaction was quenched by adding saturated sodium bicarbonate solution (10 mL). The mixture was extracted with ethyl acetate and washed with saturated sodium bicarbonate solution and brine. The organic layer was concentrated and purified by silica flash column chromatography, eluting with 20% ethyl acetate in hexanes. Fractions containing desired product were combined and concentrated to give a yellow oil.

1′,2′-dideoxy-b-1′-(N-4,5,6,7-tetrafluoroindole)-3′,5′-di-O-p-toluoyl-D-ribofuranose. (3a, 90% total yield, 95% β-epimer): ¹H NMR (CDCl₃, ppm) δ 7.96 (2H, d), 7.90 (2H, d), 7.36 (1H, d), 7.29 (2H, d), 7.24 (2H, d), 6.65 (1H, dd), 6.60 (1H, q), 5.68 (1H, m), 4.64 (2H, m), 2.72 (2H, m), 2.45 (3H, s), 2.42 (3H, s) ₁₃C-NMR (CDCl₃, ppm) δ 166.1, 144.3, 129.3, 126.5, 125.7, 115.3, 100.6, 86.8, 82.1, 74.6, 63.0, 39.4, 21.7 HRMS (FAB, 3-NBA matrix) calcd. for C₂₉H₂₃NO₅F4 (M+Na) 564.1410; found 564.1410.

1′,2′-dideoxy-b-1′-(N-indole)-3′,5′-di-O-p-toluoyl-D-ribofuranose (4a, 82% total yield, 92% β-epimer): ¹H NMR (CDCl_{3,} ppm) δ 7.97 (4H, dd), 7.62 (1H, d), 7.53 (1H, d), 7.30 (3H, m), 7.25 (2H, d), 7.15 (2H, m), 6.55 (1H, d), 6.49 (1H, dd), 5.73 (1H, m), 4.65 (2H, d), 4.57 (1H, m), 2.88 (1H, m), 2.65 (1H, ddd), 2.45 (3H, s), 2.43 (3H, s) ¹³C-NMR (CDCl₃, ppm) δ 166.2, 144.2, 129.8, 129.3, 126.7, 123.9, 122.1, 121.2, 120.4, 109.9, 103.7, 85.6, 81.6, 75.1, 64.3, 37.8, 21.7 HRMS (FAB, 3-NBA matrix) calcd. for C₂₉H₂₇NO₅ (M+) 469.1889; found 469.1867.

Procedure for deprotection of ditoluoyl-protected nucleoside. Ditoluoyl-protected nucleoside (592 mg, 1.1 mmol) was suspended in dry MeOH (9 mL) at room temperature and a 0.5 M solution of sodium methoxide (0.4 mL) was added. The reaction mixture was stirred at room temperature under inert atmosphere for 3 hours as the solution turned clear yellow void of any precipitate. The reaction was quenched with solid ammonium chloride (1.0 g) as white solid crashed out of solution. Stirring continued for 10 minutes. The liquid was decanted, washed with MeOH, and concentrated as a white solid. Silica gel chromatography ensued with 20% ethyl acetate in hexanes giving a white crystalline solid.

1′,2′-dideoxy-β-1′-(N-4,5,6,7-tetrafluoroindole)-D-ribofuranose (3b, 63% yield): ¹H NMR (CDCl₃, ppm) δ 7.36 (1H, d), 6.66 (1H, q), 6.56 (1H, t), 4.65 (1H, q), 4.02 (1H, q), 3.87 (1H, dd), 3.81 (1H, dd), 2.58 (1H, m), 2.51 (1H, m) ¹³C-NMR (CDCl₃, ppm) δ 140.4, 138.5, 138.0, 136.2 133.7, 126.3, 100.3, 86.1, 71.0, 62.2, 45.0, 41.1, 8.7 HRMS (DEI) calcd. for C₁₃H₁₁NO₃F₄ (M+) 305.0675; found 305.0663.

1′,2′-dideoxy-β-1′-(N-indole)-D-ribofuranose₄ (4b, 82% yield): ¹H NMR (CDCl₃, ppm) δ 7.61 (1H, d), 7.45 (1H, d), 7.23 (1H, t), 7.15 (2H, m), 6.55 (1H, d), 6.29 (1H, t), 4.30 (1H, m), 3.83 (1H, q), 3.60 (1H, dd), 3.53 (1H, dd), 2.43 (1H, m), 2.22 (1H, m) ¹³C-NMR (CDCl₃, ppm) δ 136.1, 129.1, 123.8, 122.2, 121.1, 120.4, 109.8, 103.5, 85.7, 84.3, 71.4, 62.3, 39.6 HRMS (DEI) calcd. for C₁₃H₁₅NO₃ (M+) 233.1052; found 233.1043.

Procedure for preparation of 5′-O-tritylated β-C-deoxynucleosides. The above-synthesized deoxynucleoside (0.08 g, 0.40 mmol)) was dissolved in a 1:1 mixture of pyridine and methylene chloride (10 mL). Diiospropylethylamine (0.10 mL, 0.60 mmol) and 4,4′-dimethoxytrityl (DMT) chloride (0.26 g, 0.80 mmol) were added to mixture and stirred for 4 hours at room temperature and then quenched with methanol (8 mL). The resulting mixture was concentrated and purified by silica gel chromatography, eluting with 20% ethyl acetate in hexanes. The product was concentrated as a yellow foam.

1′,2′-dideoxy-β-1′-(N-4,5,6,7-tetrafluoroindole)-5′-O-trityl-D-ribofuranose (3c, 74% yield): ¹H NMR (CDCl₃, ppm) δ 7.41 (2H, d), 7.34 (1H, d), 7.32-7.16 (6H, m), 6.83 (4H, m), 6.55 (3H, m), 4.56 (1H, q), 4.05 (1H, q), 3.79 (6H, s), 3.39 (2H, m), 2.49 (2H, m) ₁₃C-NMR (CDCl₃, ppm) δ 158.6, 144.5, 135.5, 130.0, 129.1, 128.1, 127.8, 127.1, 126.4, 113.2, 86.6, 85.2, 72.2, 63.5, 55.2, 41.4 HRMS (FAB, 3-NBA matrix) calcd. for C₃₄H₂₉NO₅F₄ (M+Na) 630.1880; found 630.1904.

1′,2′-dideoxy-β-1′-(N-indole)-5′-O-trityl-D-ribofuranose (4c, 95% yield): ¹H NMR (CDCl₃,ppm) δ 7.60 (1H, d), 7.50 (1H, d), 7.44 (2H, d), 7.31 (5H, m), 7.25 (5H. m), 7.17 (1H, m), 6.79 (4H, m), 6.50 (1H, d), 6.41 (1H, t), 4.61 (1H, m), 4.05 (1H, q), 3.78 (6H, s), 3.38 91H, dd), 3.30 (1H, dd), 2.64 (1H, m), 2.40 (1H, m) ₁₃C-NMR (CDCl₃, ppm) δ 158.5, 144.6, 136.0, 135.7, 130.1, 128.1, 127.9, 126.9, 124.1, 122.0, 121.0, 120.1, 113.1, 110.0, 103.1, 86.5, 84.8, 72.9, 64.0, 55.2, 40.0 HRMS (FAB, 3-NBA matrix) calcd. for C₃₄H₃₃NO₅ (M+) 535.2359; found 535.2349.

Procedure for preparation of 3′-O-phosphoramidites. The 5′-O-tritylated deoxynucleoside (0.17 g, 0.33 mmol) was dissolved in dry methylene chloride (5 mL) under _{N2} atmosphere. Diisopropylethylamine (0.25 mL, 1.4 mmol) and 2-cyanoethyl N,N-diisopropylchlorophosphoramidite (0.50 mL, 2.2 mmol) were added to the mixture and stirred for 4 hours at room temperature. The mixture was concentrated and purified by flash silica gel column chromatography, eluted with 3% triethylamine/20% ethyl acetate in hexanes. The product was obtained as an oil.

1′,2′-dideoxy-β-1′-(N-4,5,6,7-tetrafluoroindole)-5′-O-trityl-D-ribofuranose phosphoramidite (3d, 76% yield): ¹H NMR (CDCl₃, ppm) δ7.40 (2H, m), 7.31-7.21 (7H, m), 6.80 (4H, m), 6.55 (3H, m), 5.43 (1H, q), 4.65 (1H, m), 4.22 (1H, q), 3.80 (2H, m), 3.78 (6H, s) 3.61 (2H, m), 3.37 (1H, dd), 3.29 (1H, dd), 2.62 (1H, m), 2.53 (1H, m), 1.21 (2H, s), 1.17 (6H, d), 1.09 (6H, d) ¹³C-NMR (CDCl₃, ppm) δ 158.5, 144.5, 135.6, 130.1, 128.2, 127.8, 126.9, 113.1, 86.4, 73.6, 63.3, 58.0, 55.2, 43.3, 40.9, 24.5, 20.4 HRMS (FAB, 3-NBA matrix) calcd. for C₄₃H₄₆N₃O₆F₄P (M+Na) 830.2958; found 830.2982.

1′,2′-dideoxy-β-1′-(N-indole)-5′-O-trityl-D-ribofuranose phosphoramidite (4d, 67% yield): ¹H NMR (CDCl₃, ppm) δ 7.61 (1H, d), 7.56 (1H, d), 7.43 (2H, t), 7.31 (4H, m), 7.26-7.11 (6H, m). 6.76 (4H, m), 6.51 (1H, t), 5.42 (1H, m), 4.72 (1H, m), 4.23 (1H, m), 3.85 (1H, m), 3.77 (6H, s), 3.69 (2H, m), 3.60 (2H, m), 3.34 (1H, ddd), 3.26 (1H, m), 2.62 (1H, m), 2.47 (1H, m), 1.21 (2H, m), 1.19 (8H, m), 1.10 (4H, d) ¹³C-NMR (CDCl₃, ppm) δ 158.4, 144.7, 135.7, 130.2, 129.1, 128.3, 127.8, 126.8, 124.2, 121.9, 120.9, 120.1, 113.0, 110.2, 103.0, 86.3, 85.1, 70.6, 63.7, 58.3, 55.2, 43.2, 39.4, 24.6, 20.3 HRMS (FAB, 3-NBA matrix) calcd. for C₄₃H₅₁N₃O₆P (MH+) 736.3516; found 736.3517.

1a-d and 2a-d were synthesized as previously reported.

Synthesis and Characterization of Oligonucleotides.

Oligonucleotide synthesis. The deoxynucleosides 1-4 were incorporated into DNA oligonucleotides by automated solid-phase methods.5 Oligonucleotides were synthesized using an Applied Biosystems 392 DNA/RNA synthesizer in trityl-off mode using standard β-cyanoethylphosphoramidite chemistry. Lengthened coupling times (5 min) were used, giving stepwise yields of >95% by trityl monitoring. After synthesis, oligonucleotides were deprotected and removed from solid support in the usual manner. Oligomers were purified by HPLC and quantitated by UV absorption. Molar extinction coefficients were calculated by the nearest neighbor method. Values for oligonucleotides containing nonnatural residues were calculated by adding the extinction coefficient of the nonnatural nucleoside to the extinction coefficient of the core duplex. Intact structures of the oligonucleotides were confirmed by MALDI-TOF mass spectrometry.

Methods for Thermodynamic Measurements.

Thermal denaturation. Solutions for thermal denaturation studies were prepared as 1 mL samples ranging between 1 mM and 40 mM concentrations in melt buffer (1 M NaCl, 10 mM sodium phosphate, pH=7.0). The melting studies were carried out in Teflon-stoppered 1 cm pathlength quartz cells under nitrogen atmosphere on a Varian Cary 1 UV-Vis spectrophotometer equipped with a Peltier temperature controller. Absorbance was monitored at 280 nm while temperature was lowered from 90 to 0° C. at a rate of 0.5° C./minute. In all cases, the complexes displayed sharp, apparently two-state transitions.

Calculations. Melting temperature (Tm) was determined by computer fit (Meltwin 3.5) of the first derivative of absorbance with respect to 1/T. Uncertainty of Tm is estimated at ±0.5° C. based on repetitions of experiments. Free energy values were derived by two methods: (1) computer-fitting the denaturation data with an algorithm employing linear sloping baselines, using the two-state approximation for melting and (2) van't Hoff thermodynamic parameters derived from linear plots of 1/Tm vs. ln(cT) by measuring Tm at varied concentration. (Table 4 and Table 5)

Octanol-water partitioning. The experimental solvent partitioning studies for free nucleosides were carried out as described. Experiments were carried out in triplicate and the results averaged to increase accuracy. (Table 6)

TABLE S1

Thermodynamic data for duplexes containing
fluorous and hydrocarbon bases.^{a}

| | | | | | | |
|---|---|---|---|---|---|---|
| base | | | | | | |
| pair | Tₘ^{b} | ΔH°(kcal) | ΔS°(eu) | ΔG°₃₇(kcal) | ΔG°₃₇(kcal) | ΔG°₃₇(kcal) |
| (X · Y) | (° C.) | (van't Hoff) | (van't Hoff) | (van't Hoff) | (fits) | (avg.) |

^{5′}CTTTTCXTTCTT^{3′}
^{3′}GAAAAGYAAGAA^{5′}

| | | | | | | |
|---|---|---|---|---|---|---|
| B · A | 26.1 | −50.6 ± 0.8 | −142 ± 3 | −6.5 ± 0.0 | −6.4 ± 0.2 | −6.3 ± 0.1 |
| B · G | 22.8 | −44.3 ± 1.0 | −123 ± 3 | −6.2 ± 0.1 | −6.0 ± 0.1 | −6.1 ± 0.1 |
| B · C | 22.2 | −57.3 ± 2.9 | −167 ± 10 | −5.5 ± 0.2 | −5.7 ± 0.1 | −5.6 ± 0.2 |
| B · T | 22.2 | −50.7 ± 1.8 | −145 ± 6 | −5.9 ± 0.1 | −5.6 ± 0.1 | −5.8 ± 0.1 |
| ^{F}B · A | 23.9 | −57.2 ± 4.2 | −165 ± 14 | −5.9 ± 0.2 | −6.1 ± 0.0 | −6.0 ± 0.1 |
| ^{F}B · G | 27.6 | −50.1 ± 1.9 | −140 ± 6 | −6.8 ± 0.1 | −6.5 ± 0.1 | −6.7 ± 0.1 |
| ^{F}B · C | 24.5 | −53.8 ± 4.0 | −154 ± 14 | −6.0 ± 0.2 | −6.0 ± 0.1 | −6.0 ± 0.2 |
| ^{F}B · T | 25.8 | −53.2 ± 3.7 | −151 ± 12 | −6.3 ± 0.2 | −6.2 ± 0.1 | −6.3 ± 0.2 |
| I · A | 25.8 | −67.0 ± 2.3 | −197 ± 8 | −5.9 ± 0.1 | −6.5 ± 0.1 | −6.2 ± 0.1 |
| I · G | 25.1 | −49.1 ± 1.1 | −138 ± 4 | −6.4 ± 0.0 | −6.7 ± 0.2 | −6.5 ± 0.1 |
| I · C | 25.6 | −60.4 ± 3.4 | −175 ± 12 | −6.0 ± 0.1 | −6.4 ± 0.2 | −6.4 ± 0.2 |
| I · T | 24.5 | −42.9 ± 1.0 | −117 ± 3 | −6.6 ± 0.0 | −6.2 ± 0.0 | −6.4 ± 0.0 |
| ^{F}I · A | 31.8 | −51.1 ± 1.6 | −141 ± 5 | −7.5 ± 0.0 | −7.5 ± 0.0 | −7.5 ± 0.0 |
| ^{F}I · G | 31.5 | −61.9 ± 2.2 | −176 ± 7 | −7.3 ± 0.0 | −7.4 ± 0.1 | −7.3 ± 0.1 |
| ^{F}I · C | 34.6 | −55.6 ± 1.2 | −153 ± 4 | −8.0 ± 0.0 | −7.9 ± 0.1 | −7.9 ± 0.1 |
| ^{F}I · T | 30.2 | −50.5 ± 2.3 | −139 ± 7 | −7.3 ± 0.1 | −7.2 ± 0.1 | −7.2 ± 0.1 |
| B · B | 29.5 | −74.9 ± 2.5 | −220 ± 8 | −6.5 ± 0.1 | −6.7 ± 0.1 | −6.6 ± 0.1 |
| ^{F}B · ^{F}B | 30.3 | −68.5 ± 1.4 | −199 ± 5 | −6.9 ± 0.0 | −6.9 ± 0.1 | −6.9 ± 0.1 |
| I · I | 30.0 | −64.1 ± 3.5 | −185 ± 11 | −6.9 ± 0.1 | −7.3 ± 0.2 | −7.1 ± 0.2 |
| ^{F}I · ^{F}I | 40.3 | −55.6 ± 1.6 | −150 ± 5 | −9.0 ± 0.0 | −9.0 ± 0.0 | −9.0 ± 0.0 |
| T · A | 42.4 | −75.7 ± 1.8 | −213 ± 6 | −9.7 ± 0.0 | −9.8 ± 0.1 | −9.7 ± 0.1 |

^{a}Conditions: 1 M NaCl, 10 mM phosphate buffer (pH 7.0) with 0.1 mM EDTA.
^{b}Tₘ values are at 5.0 μM.

TABLE S2

Thermodynamic data for self-complementary duplexes
containing fluorous and hydrocarbon bases.^{a}

| | | | | | | |
|---|---|---|---|---|---|---|
| base | | | | | | |
| pair | Tₘ^{b} | ΔH°(kcal) | ΔS°(eu) | ΔG°₃₇(kcal) | ΔG°₃₇(kcal) | ΔG°₃₇(kcal) |
| (X · Y) | (° C.) | (van't Hoff) | (van't Hoff) | (van't Hoff) | (fits) | (avg.) |

^{5′}CGGXAGCTYCCG^{3′}
^{3′}GCCYTCGAXGGC^{5′}

| | | | | | | |
|---|---|---|---|---|---|---|
| T · A | 58.1 | −72.8 ± 3.4 | −196 ± 10 | −12.2 ± 0.2 | −11.6 ± 0.3 | −11.9 ± 0.3 |
| T · C | 20.3 | −34.7 ± 1.7 | −94 ± 6 | −5.6 ± 0.1 | −5.9 ± 0.2 | −5.7 ± 0.2 |
| B · B | 29.8 | −38.3 ± 1.5 | −102 ± 5 | −6.6 ± 0.0 | −6.7 ± 0.2 | −6.7 ± 0.1 |
| B · I | 27.8 | −32.5 ± 1.0 | −180 ± 30 | −7.4 ± 0.2 | −7.4 ± 0.2 | −7.4 ± 0.2 |
| B · ^{F}B | 30.5 | −39.8 ± 1.7 | −107 ± 6 | −6.6 ± 0.0 | −6.9 ± 0.3 | −6.8 ± 0.2 |
| B · ^{F}I | 37.2 | −35.2 ± 1.2 | −89 ± 4 | −7.5 ± 0.0 | −7.6 ± 0.1 | −7.6 ± 0.1 |
| ^{F}B · B | 32.1 | −22.2 ± 2.3 | −49 ± 7 | −7.1 ± 0.1 | −7.1 ± 0.0 | −7.1 ± 0.1 |
| ^{F}B · I | 35.1 | −39.9 ± 1.5 | −105 ± 5 | −7.3 ± 0.0 | −7.4 ± 0.2 | −7.3 ± 0.1 |
| ^{F}B · FB | 34.6 | −35.7 ± 3.0 | −92 ± 10 | −7.3 ± 0.1 | −7.3 ± 0.1 | −7.3 ± 0.1 |
| ^{F}B · FI | 41.8 | −44.2 ± 0.9 | −116 ± 3 | −8.2 ± 0.0 | −8.2 ± 0.1 | −8.2 ± 0.1 |
| I · I | 31.5 | −26.6 ± 1.9 | −63 ± 6 | −7.0 ± 0.1 | −7.3 ± 0.3 | −7.2 ± 0.2 |
| I · ^{F}I | 36.2 | −24.3 ± 1.9 | −54 ± 6 | −7.5 ± 0.1 | −7.4 ± 0.0 | −7.4 ± 0.1 |
| ^{F}I · I | 39.9 | −43.1 ± 2.9 | −113 ± 9 | −7.9 ± 0.1 | −7.9 ± 0.1 | −7.9 ± 0.1 |
| ^{F}I · ^{F}I | 45.2 | −51.8 ± 0.7 | −139 ± 2 | −8.9 ± 0.0 | −8.7 ± 0.1 | −8.8 ± 0.1 |

^{a}Conditions: 1 M NaCl. 10 mM phosphate buffer (pH 7.0) with 0.1 mM EDTA.
^{b}Tₘ values are at 5.0 μM.

TABLE S3

Thermodynamic data for self-complementary duplexes containing
dangling end fluorous and hydrocarbon bases.^{a}

| | | | | | | |
|---|---|---|---|---|---|---|
| dangling | | | | | | |
| base | Tₘ^{b} | ΔH°(kcal) | ΔS°(eu) | ΔG°₃₇(kcal) | ΔG°₃₇(kcal) | ΔG°₃₇(kcal) |
| (X) | (° C.) | (van't Hoff) | (Van't Hoff) | (Van't Hoff) | (fits) | (avg.) |

^{5′}XCGCGCG ^{3′}
^{3′} GCGCGCX^{5′}

| | | | | | | |
|---|---|---|---|---|---|---|
| B | 49.1 | −44.7 | −115 | −9.2 ± 0.1 | −9.4 ± 0.1 | −9.3 ± 0.1 |
| ^{F}B | 53.9 | −53.8 | −93 | −10.3 ± 0.2 | −10.2 ± 0.2 | −10.2 ± 0.2 |
| I | 51.6 | −60.2 | −161 | −10.2 ± 0.3 | −9.9 ± 0.1 | −10.0 ± 0.2 |
| ^{F}I | 59.3 | −52.6 | −134 | −11.1 ± 0.2 | −11.3 ± 0.2 | −11.2 ± 0.2 |

^{a}Conditions: 1 M NaCl, 10 mM phosphate buffer (pH 7.0) with 0.1 mM EDTA.
^{b}Tₘ values are at 5.0 μM.

What is claimed is:

1. A polyfluorinated nucleoside analog comprising a polyfluorinated benzene, pyrrole, pyridine, indole, isoindole, or benzoimidazole ring, or a derivative thereof.

2. A polyfluorinated nucleoside analog comprising a structure selected from the group consisting of: 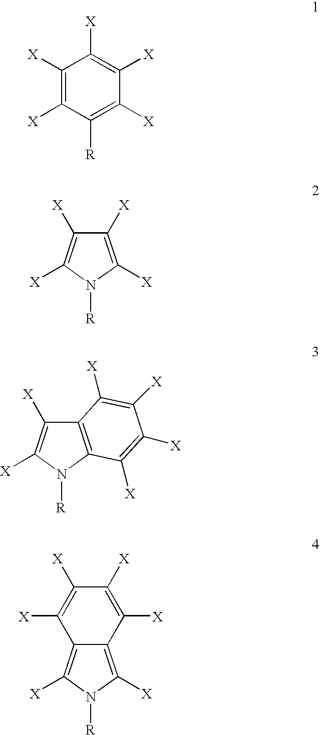
where R is a sugar moiety; and each X is independently selected from H, F, or CF₃, with the proviso that at least 3 and not more than 6 positions have fluorine-containing substituents, and wherein an annular carbon is optionally substituted with N.

3. A nucleoside analog according to claim 2, comprising a structure selected from the group consisting of:

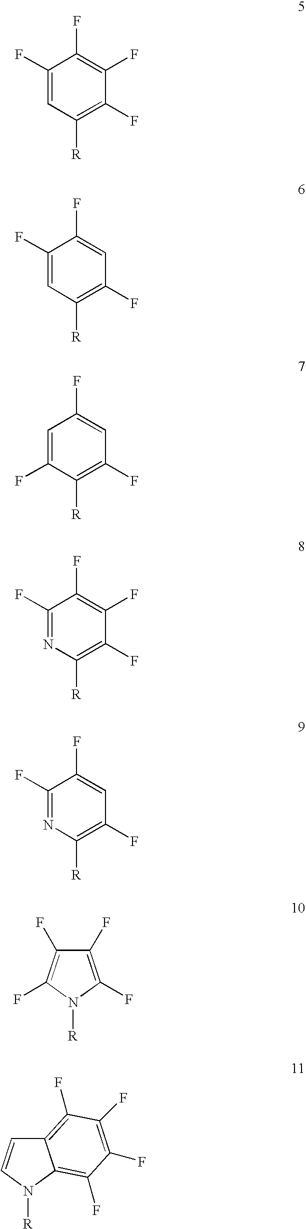

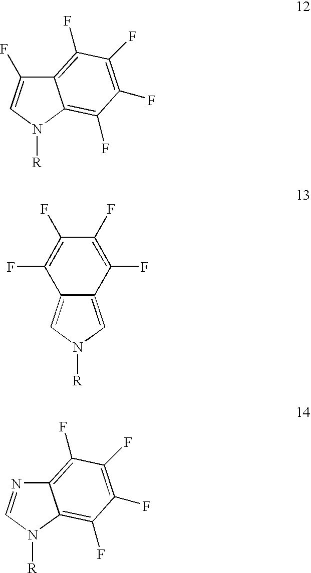

4. An oligonucleotide comprising at least one nucleoside analog according to claim 1.

5. The oligonucleotide according to claim 4, wherein said oligonucleotide is single stranded.

6. The oligonucleotide according to claim 4, wherein said oligonucleotide is double stranded.

7. The oligonucleotide according to claim 6, wherein said nucleoside analog is paired with a second polyfluorinated nucleoside analog.

8. The oligonucleotide according to claim 6, wherein said polyfluorinated nucleoside analog is mismatched with a native purine or pyrimidine base.

9. A pharmaceutical formulation comprising a polyfluorinated nucleoside analog according to claim 1, and a pharmaceutically acceptable excipient.
